SEQUENCE LISTING (13 sequences)

SEQ ID NO: 1 - DNA, 1620 bp, Clostridium perfringens
atggctaaaacattattattcggtgaagaagcaagaagatctatgcaagcgggtgtagataaattagctaacactgttaaggttacattaggaccaaaaggaagaaatgttattttagataaaaaatttggatcaccattaataacaaatgatggggttacaatagcaagagaaattgaacttgaagatgcttatgaaaatatgggagctcaacttgtaaaagaagtagctacaaagactaatgatgtggcaggagatggaactactacagctaccttattagctcaagcaattataagagaaggattaaaaaatgtaacagcaggggcaaatcctatattaataagaaatggaattaaaactgcagttgaaaaagctgtagaggaaatacaaaaaatttctaagcctgtaaatggaaaagaagacatagctagagttgctgcaatttcagcggctgatgaaaaaattggtaagctaattgcagatgctatggaaaaggtaggaaatgaaggcgttataactgtagaagaatctaaatcaatgggaactgagttagatgttgttgaaggtatgcaatttgatagaggatatgtatcagcttatatggttactgatactgaaaaaatggaagctgttttagataatccattagtattaataacagataagaaaataagcaatatacaagatttattaccattacttgagcaaatagttcaagcaggtaaaaaacttttaataatagctgatgatatagaaggcgaagctatgacaacattagttgttaataaattaagaggaacatttacttgtgttggagttaaagcacctggatttggtgatagaagaaaagaaatgttacaagatatagctactttaacaggtggcgttgttatatctgatgaagtaggcggagatttaaaagaagctacattagatatgcttggagaagctgaaagtgttaaggtaactaaagaaagtactacaatagttaatggaagaggaaactcagaagagattaaaaatagagttaaccaaataaaattacaattagaagctactacttctgaatttgacaaagaaaaattacaagaaagattagctaaattagcaggtggggttgcagtagttaaggttggagctgccactgaaacagagcttaaggaaagtaagctaagaatagaggatgctttagcagctacaaaggcagctgttgaagaaggaatagttccaggtggtggaacagcttacgtaaatgtaataaatgaagttgcaaaattaacctctgatattcaagatgaacaagttggtataaatataattgtaagatctttagaagaacctatgagacaaatagctcataatgcaggactagaaggttcagttataatagaaaaagttaaaaatagtgatgcaggtgtaggatttgatgctttaagaggagaatataaagatatgattaaagctggaatagttgatccaactaaggttacaagatcagctcttcaaaatgcagcatcagtagcatcaacattcttaacaacagaggctgctgtagcagatattccagaaaaagaaatgcctcaaggcgcaggtatgggaatggacggaatgtactaa

SEQ ID NO: 2 - DNA, 1647 bp, Escherichia coli
atggcagctaaagacgtaaaattcggtaacgacgctcgtgtgaaaatgctgcgcggcgtaaacgtactggcagatgcagtgaaagttaccctcggtccaaaaggccgtaacgtagttctggataaatctttcggtgcaccgaccatcaccaaagatggtgtttccgttgctcgtgaaatcgaactggaagacaagttcgaaaatatgggtgcgcagatggtgaaagaagttgcctctaaagcaaacgacgctgcaggcgacggtaccaccactgcaaccgtactggctcaggctatcatcactgaaggtctgaaagctgttgctgcgggcatgaacccgatggacctgaaacgtggtatcgacaaagcggttaccgctgcagttgaagaactgaaagcgctgtccgtaccatgctctgactctaaagcgattgctcaggttggtaccatctccgctaactccgacgaaaccgtaggtaaactgatcgctgaagcgatggacaaagtcggtaaagaaggcgttatcaccgttgaagacggtaccggtctgcaggacgaactggacgtggttgaaggtatgcagttcgaccgtggctacctgtctccttacttcatcaacaagccggaaactggcgcagtagaactggaaagcccgttcatcctgctggctgacaagaaaatctccaacatccgcgaaatgctgccggttctggaagctgttgccaaagcaggcaaaccgctgctgatcatcgctgaagatgtagaaggcgaagcgctggcaactctggttgttaacaccatgcgtggcatcgtgaaagtcgctgcggttaaagcaccgggcttcggcgatcgtcgtaaagctatgctgcaggatatcgcaaccctgactggcggtaccgtgatctctgaagagatcggtatggagctggaaaaagcaaccctggaagacctgggtcaggctaaacgtgttgtgatcaacaaagacaccaccactatcatcgatggcgtgggtgaagaagctgcaatccagggccgtgttgctcagatccgtcagcagattgaagaagcaacttctgactacgaccgtgaaaaactgcaggaacgcgtagcgaaactggcaggcggcgttgcagttatcaaagtgggtgctgctaccgaagttgaaatgaaagagaaaaaagcacgcgttgaagatgccctgcacgcgacccgtgctgcggtagaagaaggcgtggttgctggtggtggtgttgcgctgatccgcgtagcgtctaaactggctgacctgcgtggtcagaacgaagaccagaacgtgggtatcaaagttgcactgcgtgcaatggaagctccgctgcgtcagatcgtattgaactgcggcgaagaaccgtctgttgttgctaacaccgttaaaggcggcgacggcaactacggttacaacgcagcaaccgaagaatacggcaacatgatcgacatgggtatcctggatccaaccaaagtaactcgttctgctctgcagtacgcagcttctgtggctggcctgatgatcaccaccgaatgcatggttaccgacctgccgaaaaacgatgcagctgacttaggcgctgctggcggtatgggcggcatgggtggcatgggcggcatgatgtaa

SEQ ID NO: 3 - DNA, 1626 bp, Staphylococcus coelicolor
atggcgaagatcctgaagttcgacgaggacgcccgtcgcgccctcgagcgcggcgtcaacaagctcgccgacaccgtgaaggtgacgatcggccccaagggccgcaacgtcgtcatcgacaagaagttcggcgcccccaccatcaccaacgacggcgtcaccatcgcccgcgaggtcgaggtcgaggacccgtacgagaacctcggcgcccagctggtgaaggaggtggcgaccaagaccaacgacatcgcgggtgacggcaccaccaccgccaccgtgctcgcccaggcgctcgtgcgcgagggcctgaagaacgtcgccgccggtgcctccccggcgctgctgaagaagggcatcgacgcggccgtcgccgccgtgtcggaagaccttctcgccaccgcccgcccgatcgacgagaagtccgacatcgccgccgtggccgcgctgtccgcccaggaccagcaggtcggcgagctgatcgccgaagcgatggacaaggtcggcaaggacggtgtcatcaccgtcgaggagtccaacaccttcggtctggagctggacttcaccgagggcatggccttcgacaagggctacctgtcgccgtacttcgtgacggaccaggagcgcatggaggccgtcctcgacgacccgtacatcctgatcaaccagggcaagatctcctccatcgcggacctgctgccgctgctggagaaggtcatccaggccaacgcctccaagccgctgctgatcatcgccgaggacctggagggcgaggcgctctccaccctcgtcgtcaacaagatccgcggcaccttcaacgcggtggccgtcaaggcccccggcttcggcgaccgccgcaaggcgatgctgcaggacatggccgtcctcaccggcgccacggtcatctccgaggaggtcggcctcaagctcgaccaggtcggcctcgaggtgctcggcaccgcccgccgcatcaccgtcaccaaggacgacaccacgatcgtcgacggtgccggcaagcgcgacgaggtccagggccgcatcgcccagatcaaggccgagatcgagaacacggactccgactgggaccgcgagaagctccaggagcgcctcgcgaagctggccggcggcgtgtgcgtgatcaaggtcggcgccgccaccgaggtggagctgaaggagcgcaagcaccgtctggaggacgccatctccgcgacccgcgccgcggtcgaggagggcatcgtctccggtggtggctccgcgctggtccacgccgtcaaggtgctcgagggcaacctcggcaagaccggcgacgaggccaccggtgtcgcggtcgtccgccgcgccgcggtcgagccgctgcgctggatcgccgagaacgccggcctggagggttacgtcatcacctccaaggtcgccgacctcgacaagggccagggcttcaacgccgccaccggcgagtacggcgacctggtcaaggccggcgtcatcgacccggtgaaggtcacccgctccgccctggagaacgccgcctccatcgcctccctcctgctgacgaccgagaccctggtcgtcgagaagaaggaagaggaagagccggccgccggtggccacagccacggccactcccactga

SEQ ID NO: 4 - DNA, 1638 bp, Campylobacter jejuni
atggcaaaagaaattattttttcagatgaagcaagaaataaactttatgagggcgttaaaaaacttaatgacgcggtaaaagtaactatggggccaagaggacgcaatgttttaatccaaaaaagctttggtgctccaagcattactaaagatggcgtaagtgttgctaaagaagtagagcttaaagatagtcttgaaaatatgggtgcttcactcgtaagagaagtagcgagtaaaacagctgatcaagcaggcgatggaacaactactgcaacggttttagctcatgcaattttcaaagaaggtttaagaaatatcacagcaggtgcaaatcctatcgaggtaaaacgcggtatggataaagcttgcgaagctatagtagcagaacttaaaaaactttctcgcgaagtaaaagataaaaaagaaattgcacaagttgctacaatctcagccaactctgatgaaaaaatcggaaatttaatcgctgatgctatggaaaaagtgggcaaagatggtgttatcactgttgaagaggcaaaatcaatcaatgatgaattaaatgtagttgaaggtatgcaatttgacagaggttatttaagcccttattttatcactaatgcagaaaaaatgacagtagagctttcaagcccttatatcctgctttttgataaaaaaattacaaatttaaaagatttattaccggttttagaacaaattcaaaaaacaggcaaaccacttttaattatcgctgaagatattgaaggtgaagcgcttgcaactttggttgtaaataaacttcgcggtgttcttaatatttcagcagtgaaagctccaggttttggcgatagaagaaaagctatgcttgaagatatagcgattttaacaggtggagaagtgatttctgaagaacttggaagaactcttgaaagtgcgactatacaagatcttggacaagcttctagtgtaatcatcgataaagacaatacaaccatagtaaatggtgcaggcgaaaaagcaaatattgatgcgagagtcaatcaaatcaaagcacaaattgctgaaacaacttcagattatgacagagaaaaattacaagaaagacttgcaaaattaagtggtggtgttgcagttattaaagtaggtgcagcaactgaaactgaaatgaaagagaaaaaagatcgcgttgacgatgctttaagcgctactaaagcagcagttgaagaaggtatagtaattggtggtggtgcagcgcttatcaaagcaaaagctaaaatcaaacttgatctacagggtgatgaagcaattggcgcagctatcgttgaaagagctttaagagcacctttaagacaaattgctgaaaatgcaggatttgatgcaggtgtggttgtaaatagcgtagaaaatgctaaagatgaaaacacaggatttgatgctgcaaaaggtgaatatgttaatatgcttgaaagtggaattatcgatcctgttaaagtagaaagagtagctttactcaatgcagtttctgtagctagtatgcttttaaccacagaagcaacaattagtgaaattaaagaagataaacctactatgccagatatgagcggtatgggaggaatgggtggcatgggcggaatgatgtaa

SEQ ID NO: 5 - DNA, 1647 bp, Salmonella enterica
atggcagctaaagacgtaaaattcggtaacgacgctcgtgtgaaaatgctgcgcggcgtaaacgtactggcagatgcagtgaaagtaaccctcggtccgaaaggccgtaacgtggttctggataaatctttcggtgcgccgactatcactaaagatggtgtttccgtagcgcgtgaaatcgagctggaagacaagtttgaaaacatgggcgcgcagatggtgaaagaagttgcctctaaagcgaacgacgctgcaggcgacggcaccaccaccgcgaccgtactggcgcagtccatcattaccgaaggcctgaaagccgttgctgcgggcatgaacccgatggacctgaaacgtggtatcgacaaagcggttgctgctgcggttgaagagctgaaggcgctgtccgtaccgtgctccgactctaaagcgattgctcaggtaggtactatttccgctaactccgacgaaaccgtaggtaaactgatcgcggaagcgatggataaagtcggtaaagaaggcgtcatcactgttgaagacggtaccggtctgcaggacgaactggacgtggttgaaggtatgcagtttgaccgcggctacctgtctccttacttcatcaacaagccggaaactggcgcagtagaactggaaagcccgttcatcctgctggctgataagaaaatctccaacatccgcgaaatgctgccggttctggaagccgttgcaaaagcaggcaaaccgctgctgatcatcgctgaagatgttgaaggcgaagcgctggctaccctggtagtgaacaccatgcgtggcatcgtgaaagtggctgcggttaaagcaccgggcttcggcgatcgtcgtaaggcgatgctgcaggatatcgctaccctgaccggcggtaccgtaatctctgaagagatcggtatggagctggaaaaagcaaccctggaagacctgggtcaggcgaaacgtgttgtgatcaacaaagacaccaccaccatcattgatggcgtgggtgaagaagctgccatccagggccgtgttgctcagatccgtcagcagattgaagaagcgacctccgactacgatcgtgaaaaactgcaggagcgcgtagcgaaactggcaggcggcgttgcggtaatcaaagttggcgctgcgaccgaagttgaaatgaaagagaagaaagcccgcgttgaagatgccctgcacgcgacccgtgctgcggtagaagaaggcgtggttgctggtggtggcgttgcgctgatccgcgttgcttctaaaattgctgacctgaaaggccagaacgaagaccagaacgtgggtatcaaagttgcgctgcgcgcaatggaagctccgctgcgtcagatcgtgctgaactgcggcgaagagccgtctgttgtcgctaacaccgttaaaggcggcgacggtaactacggttacaacgcagcaactgaagaatacggcaacatgatcgatatgggtatcttggacccaaccaaagttacccgttctgcgctgcaatacgcggcttctgtggctggtctgatgatcactaccgagtgcatggtgaccgacctgccgaaaagcgatgctcctgatttaggcgctgctggcggcatgggtggtatgggtggtatgggcggcatgatgtaa

SEQ ID NO: 6 - DNA, 26 bp, Artificial Sequence
  Synthetic
gannnngcnggngayggnacnacnac

SEQ ID NO: 7 - DNA, 26 bp, Artificial Sequence
  Synthetic
yknykntcnccraanccnggngcytt

SEQ ID NO: 8 - DNA, 28 bp, Artificial Sequence
  Synthetic
gacaaagtcggtaaagaaggcgttatca

SEQ ID NO: 9 - DNA, 26 bp, Artificial Sequence
  Synthetic
agccgttgcaaaagcaggcaaaccgc

SEQ ID NO: 10 - DNA, 25 bp, Artificial Sequence
  Synthetic
ttgagcaaatagttcaagcaggtaa

SEQ ID NO: 11 - DNA, 25 bp, Artificial Sequence
  Synthetic
gcaactctggttgttaacaccatgc

SEQ ID NO: 12 - DNA, 25 bp, Artificial Sequence
  Synthetic
tggagaaggtcatccaggccaacgc

SEQ ID NO: 13 - DNA, 25 bp, Artificial Sequence
  Synthetic
tagaacaaattcaaaaaacaggcaa

(19) United States
(12) Patent Application Publication
(10) Pub. No.: US 2004/0101826 A1
(43) Pub. Date: May 27, 2004

(54) Monitoring high-risk environments

(76) Inventors: Alison M. Jones, Eden Prairie, MN (US); W. Wade Robey, Excelsior, MN (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/392,041

(22) Filed: Mar. 18, 2003

Related U.S. Application Data
(63) Continuation-in-part of application No. 10/306,113, filed on Nov. 27, 2002.

(51) Int. Cl. C12Q 1/70; C12Q 1/68; G01N 33/554; G01N 33/569; G06F 19/00; G01N 33/48; G01N 33/50

(52) U.S. Cl. 435/5; 435/6; 435/7.32; 702/19

(57) ABSTRACT

The invention provides for methods of monitoring high-risk environments for the presence or absence of microbes by detecting the presence or absence of a cpn60 marker in a sample.

### CROSS-REFERENCE TO RELATED APPLICATIONS

This application is a continuation-in-part of U.S. Ser. No. 10/306,113, filed Nov. 27, 2002.

### TECHNICAL FIELD

This invention relates to monitoring high-risk environments for microbes, including microbial pathogens, and more particularly to detecting microbes in high-risk environments by detecting microbial markers.

### BACKGROUND

From a public health perspective, the detection of microbes, particularly microbial pathogens, in environments such as food processing facilities and health care institutions, is critically important. For example, nosocomial outbreaks, or infectious outbreaks that occur in excess of generally 2 times the normal expectancy in patients in health care institutions, present difficult issues of associated morbidity, mortality, and expense in a health care system already crippled by rising insurance and health care costs. The CDC has reported that nearly $5 billion are added to U.S. health costs every year as a result of nosocomial infections. Similarly, according to the CDC's National Nosocomial Surveillance System, the rate of hospital-related fungal infections nearly doubled between 1980 and 1990. In 1997, an estimated 240,000 individuals showed clinical symptoms of endemic mycoses, with the mortality rate in patients with systemic fungal infections ranging from 30-100%, depending on the pathogen. Over-reliance on antibiotics has led to antibiotic resistant bacterial strains, themselves often of nosocomial origin. Finally, pathogen contamination in environments such as food processing plants and day-care centers has led to widespread infectious outbreaks, often with intensive and expensive investigation required on the local or national level to determine and control the source of the pathogen. Thus, knowledge of the nature and amount of a microbial pathogen in a particular environment may be required for efficient infectious disease source identification, outbreak management, and treatment.

Microbial profiles are representations of individual strains, subspecies, species, and/or genera of microorganisms within a community of microorganisms. Generally, determining a microbial profile involves taxonomic and/or phylogenetic identification of the microbes in a community. A microbial profile also can include quantitative information about one or more members of the community. Once one or more microorganisms have been identified in a microbial community, microbial profiles can be presented as, for example, lists of microorganisms, graphical or tabular representations of the presence and/or numbers of microorganisms, or any other appropriate representation of the diversity and/or population levels of the microorganisms in a community. Microbial profiles are useful for identifying pathogenic and non-pathogenic microbial organisms in biological and non-biological samples (e.g., samples from animals, the environment, or inanimate objects).

A microbial profile can be determined using any of a number of known methods. For example, the microbes in a sample can be cultured and colonies identified and/or enumerated. It has been estimated, however, that culturing typically recovers only about 0.1% of the microbial species in a sample (based on comparisons between direct microscopic counts and recovered colony-forming units). Culture-independent methods to determine microbial profiles can include extracting and analyzing microbial macromolecules from a sample. Useful target molecules typically include those that as a class are found in all microorganisms, but are diverse in their structures and thereby reflect the diversity of the microbes. For example, various nucleic acid-based assays can be employed to determine a microbial profile. Some nucleic acid-based population methods use denaturation and reannealing kinetics to derive an indirect estimate of the guanine and cytosine (% G+C) content of the DNA in a sample, for example. The % G+C technique provides an overall view of the microbial community, but typically is sensitive only to massive changes in the make-up of the community.

Genetic fingerprinting is another nucleic-acid based method that can be used to determine a microbial profile. Genetic fingerprinting utilizes random-sequence oligonucleotide primers that hybridize specifically to random sequences throughout the genome. Amplification results in a multitude of products, and the distribution of those products is referred to as a genetic fingerprint. Particular patterns can be associated with a community of microbes in the sample. Genetic fingerprinting, however, lacks the ability to conclusively identify specific microbial species.

Denaturing or temperature gradient gel electrophoresis (DGGE or TGGE) is another nucleic acid-based technique that can be used to determine a microbial profile. As amplification products are electrophoresed in gradients with increasing denaturant or temperature, the double-stranded molecule melts and its mobility is reduced. The melting behavior is determined by the nucleotide sequence, and unique sequences will resolve into individual bands. Thus, a D/TGGE gel yields a genetic fingerprint characteristic of the microbial community, and the relative intensity of each band reflects the abundance of the corresponding microorganism. An alternative format includes single-stranded conformation polymorphism (SSCP). SSCP relies on the same physical basis as % G+C renaturation methods, but reflects a significant improvement over such methods.

In addition, a microbial profile can be determined using terminal restriction fragment length polymorphism (TRFLP). Amplification products can be analyzed for the presence of known sequence motifs using restriction endonucleases that recognize and cleave double-stranded nucleic acids at these motifs. Alternative approaches include “amplified ribosomal DNA restriction analysis (AADRA)” in which the entire amplification product, rather than just the terminal fragment, is considered.

A microbial profile also can be determined by cloning and sequencing microbial nucleic acids present in a biological or non-biological sample (e.g., a biological sample from an animal). Cloning of individual nucleic acids into Escherichia coli and sequencing each nucleic acid gives the highest density of information but requires the most effort. Although sequencing nucleic acids is automated, routine monitoring of changes in the microbial profile of an animal by cloning and sequencing nucleic acids from the microorganisms still requires considerable time and effort.

Therefore, despite the existence of methods for determining microbial profiles, there remains a need for rapid, sensitive, and quantitative methods capable of detecting and identifying microbes, particularly microbial pathogens, in high-risk environments.

### SUMMARY

The invention is based on the discovery that the presence or absence of a microbe in a high-risk environment can be determined quickly and sensitively by detecting the presence and/or concentration of a microbial marker, specifically a cpn60 marker, in a sample obtained from the high-risk environment. Chaperonin 60 (cpn60) markers are particularly useful for determining the presence of a microbe in a sample and optionally determining a microbial profile of a sample. Chaperonin proteins are molecular chaperones required for proper folding of polypeptides in vivo. cpn60 is found universally in prokaryotes and in the organelles of eukaryotes, and can be used as a species-specific target and/or probe for identification and classification of microorganisms. Sequence diversity within this protein-encoding gene appears greater between and within bacterial genera than for 16S rDNA sequences, thus making cpn60 a superior target sequence having more distinguishing power for microbial identification at the species level than 16S rDNA sequences.

Accordingly, the detection of the presence and/or concentration of the cpn60 marker may be capable of providing a microbial profile of the sample. In particular, microbial profiles of biological and non-biological samples from a high-risk environment can be determined using methods that involve detection of cpn60 markers, including cpn60-specific nucleic acid molecules and cpn60-specific polypeptides.

Methods of the invention are rapid and sensitive, and can be used to detect the presence or absence of cpn60-containing microbes in general, as well as to identify what species of microbes are present and in what amounts. Using cpn60 primers, probes, and antibodies, methods of the invention can include amplifying cpn60-specific nucleic acids and detecting amplification products using techniques such as fluorescence resonance energy transfer (FRET). Other rapid and sensitive methods for detecting and quantifying cpn60-specific nucleic acids include fluorescent in situ hybridization (FISH), for example. Accordingly, primers and probes for detecting cpn60-containing microbial species are provided by the invention, as are methods for using such primers and probes and kits containing such primers and probes. Similarly, the invention also provides methods for detecting cpn60-specific polypeptides, such as enzyme-linked immunoassays (ELISAs), or other polypeptide detection methods, including surface plasmon resonance techniques, mass spectrometry, and electrophoretic methods. Accordingly, kits containing cpn60-specific antibodies are also contemplated by the present invention.

In one aspect, the invention provides methods for monitoring a high-risk environment for the presence or absence of one or more microbes. Such a method includes providing a sample obtained from the high-risk environment; and detecting the presence or absence of a cpn60 marker in the sample. Generally, the presence of the cpn60 marker is indicative of the presence of the one or more microbes.

In one embodiment, the detecting step is capable of providing a microbial profile of the sample. Typically, the microbial profile includes identifying one or more microbes in the sample, and may further include quantifying the amount of one or more microbes in the sample. In addition, a microbial profile of the high-risk environment can be acquired and compared at two or more points, for example, time points or location points. Further, a control microbial profile from a control sample can be acquired from the high-risk environment, which can be compared to the sample microbial profile.

Generally, the cpn60 marker is a cpn60-specific nucleic acid or a cpn60-specific polypeptide. In one embodiment, the cpn60-specific nucleic acid is a genomic nucleic acid coding sequence of a cpn60 protein, for example, of chromosomal origin. In another embodiment, the cpn60-specific nucleic acid is an amplified sequence of a cpn60 coding sequence of the microbe.

Generally, the detecting step can be a nucleic acid-based assay or a polypeptide-based assay. Representative nucleic acid-based assays include PCR and FISH assays, while representative polypeptide-based assays include an immunodiagnostic assay (e.g., ELISA), a mass spectrometric technique, and a surface plasmon resonance technique.

Typically, a microbe that can be detected by methods of the invention include bacteria, protozoa, rickettsiae, and fungi. Representative bacterial microbes include the Staphylococcus, Streptococcus, Pseudomonas, Escherichia, Bacillus, Brucella, Chlamydia, Clostridium, Shigella, Mycobacterium, Agrobacterium, Bartonella, Borellia, Bradyrhizobium, Ehrlichia, Haemophilus, Helicobacter, Heliobacter, Lactobacillus, Neisseria, Rhizobium, Streptomyces, Synechococcus, Zymomonas, Synechocyotis, Mycoplasma, Yersinia, Vibrio, Burkholderia, Franciscella, Legionella, Salmonella, Bifidobacterium, Enterococcus, Enterobacter, Citrobacter, Bacteroides, Prevotella, Xanthomonas, Xylella, and Campylobacter genera. Representative protozoan microbes include Acanthamoeba, Cryptosporidium, and Tetrahymena genera. Representative fungal microbes include Aspergillus, Colletrotrichum, Cochliobolus, Helminthosporium, Microcyclus, Puccinia, Pyricularia, Deuterophoma, Monilia, Candida, and Saccharomyces. Representative rickettsiae microbes include Coxiella burnetti, Bartonella quintana, Rochlimea Quintana, Rickettsia Quintana, Rickettsia prowasecki, and Rickettsia rickettsii.

Examples of high-risk environments include a retail food industry facility, a school, a medical environment, a water facility, a residence, a food transport vehicle, a processing facility, or a research facility. For example, a retail food industry facility can be a butcher shop, a grocery store, a restaurant, a cafeteria, an entertainment facility (e.g., a theater, a park, a zoo, a rink, an arena, a civic center, a museum, or a stadium), or a convenience store; a medical environment can be a hospital, a physician's office, a dental office, a clinic, a nursing home, an outpatient facility, a physical therapy facility, a spa, an operating room, or a medical diagnostic laboratory; a water facility can be a wastewater treatment plant, a potable water facility, a desalinization facility, a recycled water facility, an aquaculture facility, an air conditioning unit, a humidifier, a water storage tank, a water fountain, a fire hydrant, a tub, a hot tub, a sauna, a steam bath, or a water tap; a food transport vehicle can be a truck, a rail car, or a ship; and a processing facility can be a food processing facility (e.g., an abbatoir, a packaging facility, a purification plant, or a fermentation vessel), a chemical processing facility, or a biological processing facility.

In one embodiment, the sample is a tissue sample. A tissue sample can be a biopsy sample, or can be derived from a swab of an animal. Representative animals include a human, a cow, a pig, a horse, a goat, a sheep, a dog, a cat, a bird, a monkey, a fish, a clam, an oyster, a mussel, a lobster, a shrimp, and a crab. Representative tissue samples from such animals include an eye, a tongue, a cheek, a hoof, a beak, a snout, a foot, a hand, a mouth, a teat, the gastrointestinal tract, a feather, an ear, a nose, a mucous membrane, a scale, a shell, the fur, and the skin.

In another embodiment, the sample is a fomite or is derived from a fomite present in the high-risk environment.

In another embodiment, the sample is a food sample such as a prepared food sample, a raw food sample, a cooked food sample, or a perishable food sample. Representative food samples include beef, pork, poultry, seafood, dairy (milk, eggs, or cheese), fruit, vegetable, seed, nut, and fungus. Further, the sample can be a liquid sample such as a water sample, a blood sample, a urine sample, a lachrymal sample, a sweat sample, a saliva sample, a lymph sample, and a cerebrospinal fluid sample.

In another aspect, the invention provides an article of manufacture. An article of manufacture of the invention includes at least one cpn60 antibody, wherein the cpn60 antibody is attached to a solid support; and an indicator molecule. An article of manufacture also can include instructions for using the cpn60 antibody to detect a cpn60-containing microbe. A representative solid support is a dipstick.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

FIG. 1 is the sequence of a cpn60 gene from Clostridium perfringens (SEQ ID NO:1; GenBank® Accession No. NC_{—}003366). Sequences to which the universal cpn60 primers described herein can hybridize (or the complement thereof) are underlined.

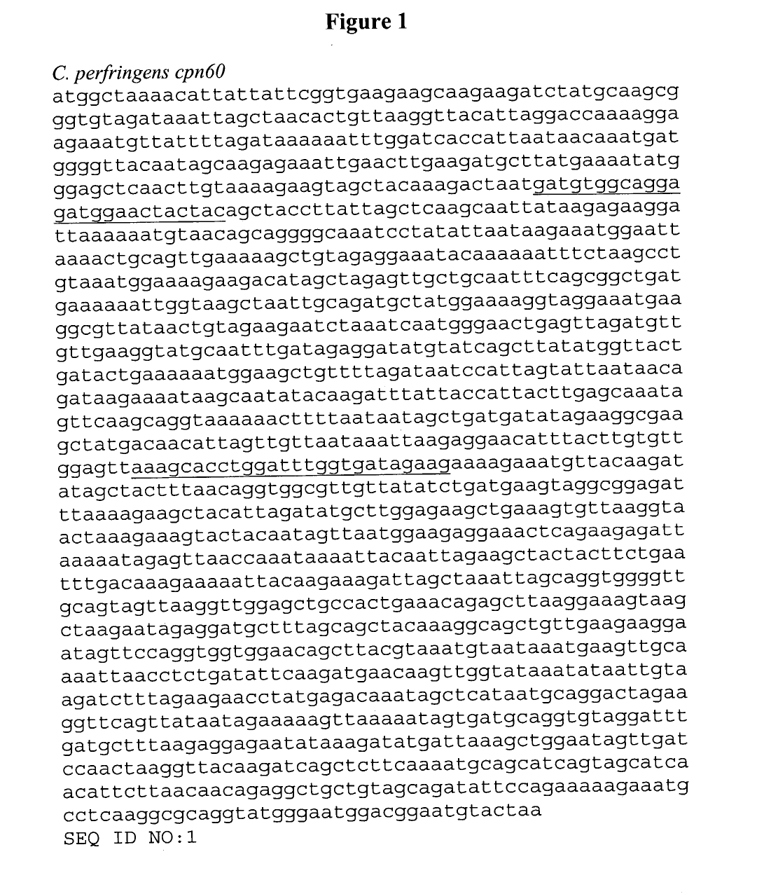

FIG. 2 is the sequence of a cpn60 gene from Escherichia coli (SEQ ID NO:2; GenBank® Accession No. NC_{—}000913). Sequences to which the universal cpn60 primers described herein can hybridize (or the complement thereof) are underlined.

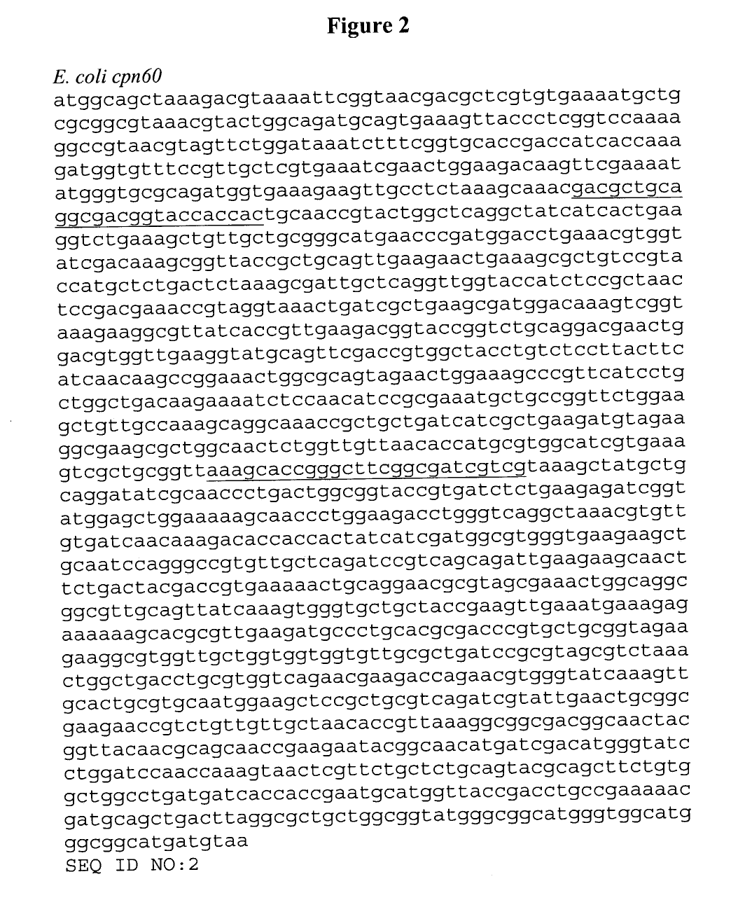

FIG. 3 is the sequence of a cpn60 gene from Staphylococcus coelicolor (SEQ ID NO:3; GenBank® Accession No. AL939121). Sequences to which the universal cpn60 primers described herein can hybridize (or the complement thereof) are underlined.

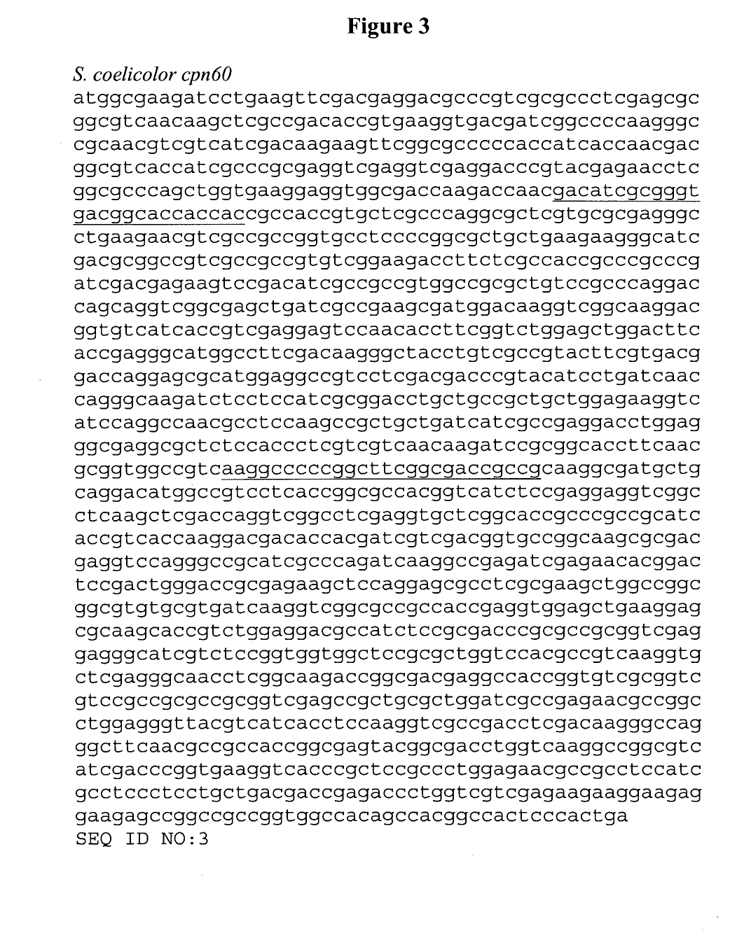

FIG. 4 is the sequence of a cpn60 gene from Campylobacter jejuni (SEQ ID NO:4; GenBank® Accession No. NC_{—}002163). Sequences to which the universal cpn60 primers described herein can hybridize (or the complement thereof) are underlined.

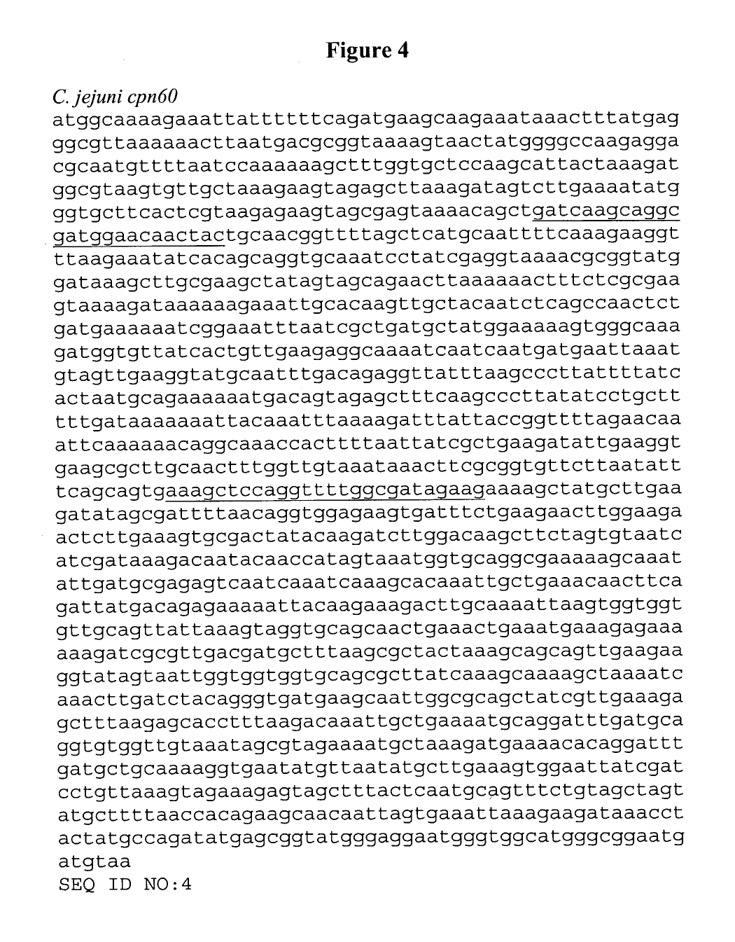

FIG. 5 is the sequence of a cpn60 gene from Salmonella enterica (SEQ ID NO:5; GenBank® Accession No. NC_{—}003198). Sequences to which the universal cpn60 primers described herein can hybridize (or the complement thereof) are underlined.

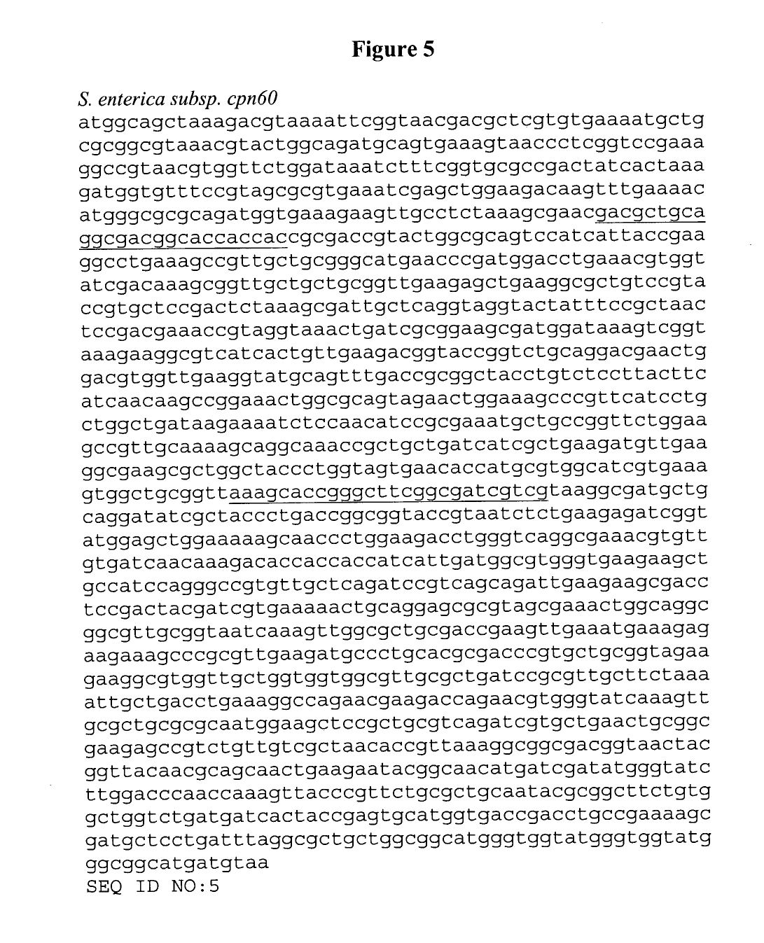

Like reference symbols in the various drawings indicate like elements.

### DETAILED DESCRIPTION

The present invention provides methods for monitoring high-risk environments for the presence or absence of one or more microbes. The microbes may be pathogens. In particular, the presence or absence of a microbe in a high-risk environment can be determined quickly and sensitively by detecting the presence and/or concentration of a microbial marker, specifically a cpn60 marker, in a sample obtained from the high-risk environment.

High-Risk Environments

As used herein, a high-risk environment is an environment at risk of contamination by one or more microbes because of the nature of the activity conducted therein or because of the environment's potential to be a source of a microbe. High-risk environments include, but are not limited to, the following: a retail food industry facility, a school, a medical environment, a water facility, a residence, perishable foods or improperly preserved foods, a transportation facility, a processing facility, and a research facility. For example, the retail food industry has traditionally been a source of serious infectious outbreaks, and is an example of one type of high-risk environment. Retail food industry locations includes such places as a butcher shop, a grocery store, a restaurant, a cafeteria, an entertainment facility, and a convenience store.

Entertainment facilities include such places as theaters, libraries, malls, parks, zoos, rinks, arenas, civic centers, museums, amusement parks, arcades, athletic fields and locations, conference halls, meeting rooms, and stadiums. Entertainment facilities may be high-risk environments because, for example, food is processed, prepared, and sold on-site and because of the likelihood of inoculating a large number of humans in such locations.

Medical environments are also examples of high-risk environments. Medical environments generally have a close physical association of numerous patients with a variety of illnesses, many of who are already in an immunocompromised state. The potential for cross-contamination, nosocomial outbreaks, and the development of antibiotic resistant strains is high in such an environment. Nonlimiting examples of medical environments include a hospital, a physician's office, a dental office, a clinic, a nursing home, an outpatient facility, a physical therapy facility, a spa, an operating room, and a medical diagnostic laboratory.

Water facilities are yet additional examples of high-risk environments. For example, wastewater treatment plants are naturally confronted with a variety of pathogens in the water to be treated. Aquaculture facilities, such as fish farms, oyster beds, etc., are also susceptible to infectious outbreaks. Air conditioning units have faced increased scrutiny as a source of infectious agents after the Legionnaire's outbreak. Hot tubs have been recently implicated as a source of Mycobacterium avium infections (“hot tub lung”) in people who use them frequently. Other examples of water facilities include potable water facilities, desalinization facilities, dams, recycled water facilities, humidifiers, water storage tanks, potable water reservoirs (e.g., water coolers), water fountains, fire hydrants, tubs, saunas, steam baths, and water taps.

Transportation facilities are additional examples of high-risk environments. A transportation facility may be high-risk because it is used, e.g., to transport food. For example, food transport vehicles such as railway cars, trucks, tank cars, and shipping vessels that transport bulk quantities of food may need to be monitored prior to loading and after off-loading. Alternatively, a transportation facility may be a high-risk environment because of the potential for a microbe to be carried through such a facility. Non-limiting examples include a car, a bus, a plane, a train, a bicycle, a motorcycle, a ship, an airport, a bus terminal, a train terminal, a port, a Custom's checkpoint, and an immigration checkpoint. For example, contaminated food (e.g., fruit) may be carried through an immigration checkpoint.

Processing facilities, including food, chemical, and biological processing facilities, are other examples of high-risk environments. Food processing facilities have been under increasing pressure to control microbial contamination of processed foods, such as by requiring the implementation of Hazard Analysis and Critical Control Point Plans (HACCP) and antimicrobial intervention techniques. Food processing facilities include abbatoirs (slaughter-houses), packaging facilities, purification (e.g. radiation, pasteurization, fumigation) facilities, storage locations (e.g., silos, vessels, tanks), and fermentation vessels. Chemical and biological processing facilities can include analytical laboratories, production plants, pilot plants, and purification plants.

Foods, particularly perishable foods and improperly preserved, stored, or handled foods, are also examples of high-risk environments. Perishable foods include, for example, milk, eggs, cheeses, breads, buffet table menu items, carry-out menu items, vegetables, and fruits. Improperly preserved foods include those that are commercially preserved (e.g., canned, sealed, jarred, bagged etc. by a commercial food source) or self-preserved (e.g., home canning, etc.). The food may be prepared, e.g., in a restaurant or a home kitchen. Such a prepared food sample may be either cooked or raw (e.g., salads, juices, fruits). Alternatively, the food may be unprocessed. Typical food products include beef, pork, poultry, seafood, dairy, fruit, vegetable, seed, nut, fungus, and grain. Dairy food samples include milk, eggs, butter, and cheese, as well as condiments and sauces prepared from such dairy foods (e.g., mayonnaise, aioli, cream sauces, hollandaise sauces, etc.).

Sample Types and Sampling Methods

The methods described herein are capable of detecting the presence or absence of a microbe, and optionally a microbial profile, based on the presence of a cpn60 marker in a sample obtained from a high-risk environment. Microbial profiles can be determined for biological or non-biological samples.

As used herein, “biological sample” refers to any sample obtained, directly or indirectly, from a subject animal or control animal. Representative biological samples that can be obtained from an animal include or are derived from biological tissues, biological fluids, and biological elimination products (e.g., feces). Biological tissues can include biopsy samples or swabs of the biological tissue of interest, e.g., nasal swabs, throat swabs, dermal swabs. The tissue can be any appropriate tissue from an animal, such as a human, cow, pig, horse, goat, sheep, dog, cat, bird, monkey, fish, clam, oyster, mussel, lobster, shrimp, and crab. Depending on the microbe and the type of high-risk environment, the tissue of interest to sample (e.g., by biopsy or swab) can be an eye, a tongue, a cheek, a hoof, a beak, a snout, a foot, a hand, a mouth, a teat, the gastrointestinal tract, a feather, an ear, a nose, a mucous membrane, a scale, a shell, the fur, and the skin.

Biological fluids can include bodily fluids (e.g., urine, milk, lachrymal fluid, vitreous fluid, sputum, cerebrospinal fluid, sweat, lymph, saliva, semen, blood, or serum or plasma derived from blood); a lavage such as a breast duct lavage, lung lavage, a gastric lavage, a rectal or colonic lavage, or a vaginal lavage; an aspirate such as a nipple or teat aspirate; a fluid such as a cell culture or a supernatant from a cell culture; and a fluid such as a buffer that has been used to obtain or resuspend a sample, e.g., to wash or to wet a swab in a swab sampling procedure. Biological samples can be obtained from an animal using methods and techniques known in the art. See, for example, Diagnostic Molecular Microbiology: Principles and Applications (Persing et al. (eds), 1993, American Society for Microbiology, Washington D.C).

Biological samples also can be obtained from the environment (e.g., air, water, or soil). Methods are known for extracting biological samples (e.g., cells) from such environments. Additionally, a biological sample suitable for use in the methods of the invention can be a substance that one or more animals have contacted. For example, an aqueous sample from a water bath, a chill tank, a scald tank, or other aqueous environments with which a subject or control animal has been in contact, can be used in the methods of the invention to evaluate a microbial profile. A soil sample that one or more subject or control animals have contacted, or on which an animal has deposited fecal or other biological material, also can be used in the methods of the invention. For example, nucleic acids can be isolated from such biological samples using methods and techniques known in the art. See, for example, Diagnostic Molecular Microbiology: Principles and Applications (Persing et al. (eds), 1993, American Society for Microbiology, Washington D.C).

The methods of the present invention can also be used to detect the presence of microbes and/or microbial pathogens in or on non-biological samples. For example, a fomite present in a high-risk environment may be sampled to detect the presence or absence of a microbe. A fomite is a physical (inanimate) object that serves to transmit, or is capable of transmitting, an infectious agent, e.g., a microbial pathogen, from animal to animal. (It is noted that inanimate objects such as food, air, and liquids are not considered fomites, but are considered infectious “vehicles,” or media that are routinely taken into the body.) Indeed, one study that evaluated the presence of Salmonella spp., Listeria spp., and Yersinia spp. pathogenic microbes on various abbatoir fomites detected Salmonella spp. on 11.1% of meat cleavers, 6.25% of worktables, and 5.6% of floors; Yersinia enterocolitica was found on 16.7% of slaughter floors and on 12.5% of worktables; and Listeria monocytogenes was isolated from 13.3% of cold room floor swabs and on 7.1% of hand-wash basins. See Kathryn Cooper, Guelph Food Technology Centre, “The Plant Environment Counts: Protect your Product through Environmental Sampling,” Meat & Poultry, May 1999. Non-limiting examples of fomites include utensils, knives, drinking glasses, food processing equipment, cutting surfaces, cutting boards, floors, ceilings, walls, drains, overhead lines, ventilation systems, waste traps, troughs, machines, toys, storage boxes, toilet seats, door handles, clothes, gloves, bedding, combs, shoes, changing tables (e.g., for diapers), diaper bins, toy bins, food preparation tables, food transportation vehicles (e.g., rail cars and shipping vessels), gates, ramps, floor mats, foot pedals of vehicles, sinks, washing facilities, showers, tubs, buffet tables, surgical equipment and instruments, and analytical instruments and equipment.

A microbe may be left as a residue on a fomite. In such cases, it is important to detect accurately the presence of the pathogen on the fomite in order to prevent the spread of the pathogen. For example, it is known that microbes may exist in viable but nonculturable forms on fomites, or that nonculturable bacteria of selected species can be resuscitated to a culturable state under certain conditions. Often such nonculturable bacteria are present in biofilms on fomites. Accordingly, detection methods that rely on culturable forms may significantly under-report microbial contamination on fomites. The methods of the present invention, including PCR-based methods, can aid in the detection of microbes, particularly nonculturable forms, by amplification and detection of cpn60-specific nucleic acid sequences.

The sample from the high-risk environment can also be a food sample. For example, the sample may be a prepared food sample, e.g., from a restaurant. Such a prepared food sample may be either cooked or raw (e.g., salads, juices, fruits). In other embodiments, the food sample may be unprocessed and/or raw, e.g., a tissue sample of an animal from a slaughterhouse, either prior to or after slaughter. The food sample may be perishable. Typically food samples will be taken from food products such as beef, pork, poultry, seafood, dairy, fruit, vegetable, seed, nut, fungus, and grain. Dairy food samples include milk, eggs, butter, and cheese, as well as sauces and condiments made from the same.

Methods for collecting and storing biological and non-biological samples are generally known to those of skill in the art. For example, the Association of Analytical Communities International (AOAC International) publishes and validates sampling techniques for testing foods and agricultural products for microbial contamination. See also WO 9832020 (PCT/WO 97° U.S04289) and U.S. Pat. No. 5,624,810, which set forth methods and devices for collecting and concentrating microbes from the air, liquid, or a surface. WO 9832020 also provides methods for removing somatic cells, or animal body cells present at varying levels in certain samples.

In particular embodiments of the methods described herein, a separation and/or concentration step may be necessary to separate any microbes present from other components of a sample or to concentrate the microbe to an amount sufficient for rapid detection. For example, a sample suspected of containing a biological microbe may require a selective enrichment of the microbe (e.g., by culturing in appropriate media, e.g., for 4-96 hours, or longer) prior to employing the detection methods described herein. Alternatively, appropriate filters and/or immunomagnetic separations can concentrate a microbe without the need for an extended growth stage. For example, antibodies specific for a cpn60-specific polypeptide can be attached to magnetic beads and/or particles. Multiplexed separations, in which two or more concentration processes are employed, are also contemplated, e.g., centrifugation, membrane filtration, electrophoresis, ion exchange, affinity chromatography, and immunomagnetic separations.

Certain air or water samples may need to be concentrated. For example, certain air sampling methods require the passage of a prescribed volume of air over a filter to trap any microbes, followed by isolation into a buffer or liquid culture. Alternatively, the focused air is passed over a plate (e.g., agar) medium for growth of any microbe.

Methods for sampling a tissue or a fomite with a swab are known to those of skill in the art. Generally, a swab is hydrated (e.g., with an appropriate buffer, such as Cary-Blair medium, Stuart's medium, Amie's medium, PBS, buffered glycerol saline, or water) and used to sample an appropriate surface (a fomite or tissue) for a microbe. Any microbe present is then recovered from the swab, such as by centrifugation of the hydrating fluid away from the swab, removal of supernatant, and resuspension of the centrifugate in an appropriate buffer, or by washing of the swab with additional diluent or buffer. The so-recovered sample may then be analyzed according to the methods described herein for the presence of a microbe. Alternatively, the swab may be used to culture a liquid or plate (e.g., agar) medium in order to promote the growth of any microbes for later testing. Suitable swabs include both cotton and sponge swabs; see, for example, those provided by Tecra®, such as the Tecra ENVIROSWAB®.

The samples from the high-risk environment can be used “as is,” or may need to be treated prior to application of the detection methods employed herein. For example, samples can be processed (e.g., by nucleic acid or protein extraction methods and/or kits known in the art) to release nucleic acid or proteins. In other cases, a biological sample can be contacted directly with PCR reaction components and appropriate oligonucleotide primers and probes.

Detection of cpn60 Markers

Methods provided herein are useful for determining the presence of one or more microbes and/or microbial pathogens in a high-risk environment and optionally provide microbial profiles of the high-risk environment. As used herein, “microbes” refers to bacteria, protozoa, rickettsiae, and fungi. Microbial communities for which a microbial profile can be generated can include but are not limited to the following examples of prokaryotic genera: Staphylococcus, Streptococcus, Pseudomonas, Escherichia, Bacillus, Brucella, Chlamydia, Clostridium, Shigella, Mycobacterium, Agrobacterium, Bartonella, Borellia, Bradyrhizobium, Ehrlichia, Haemophilus, Helicobacter, Heliobacter, Lactobacillus, Neisseria, Rhizobium, Streptomyces, Synechococcus, Zymomonas, Synechocyotis, Mycoplasma, Yersinia, Vibrio, Burkholderia, Franciscella, Legionella, Salmonella, Bifidobacterium, Enterococcus, Enterobacter, Citrobacter, Bacteroides, Prevotella, Xanthomonas, Xylella, and Campylobacter; the following examples of protozoa genera: Acanthamoeba, Cryptosporidium, and Tetrahymena; the following examples of fungal genera: Aspergillus, Colletrotrichum, Cochliobolus, Helminthosporium, Microcyclus, Puccinia, Pyricularia, Deuterophoma, Monilia, Candida, and Saccharomyces; and the following rickettsiae microbes: Coxiella burnetti, Bartonella quintana, Rochlimea Quintana, Rickettsia Quintana, Rickettsia prowasecki, and Rickettsia rickettsii.

The detection of a microbe or microbial profile in a sample (e.g., a biological sample or a non-biological sample) obtained from a high-risk environment can be determined using methods that involve detection of a cpn60 marker. cpn60 markers include cpn60-specific nucleic acids and cpn60-specific polypeptides. As used herein, a cpn60-specific nucleic acid is a nucleic acid that includes, is complementary to, or specifically hybridizes to all or a portion of the genomic cpn60 nucleic acid sequence. Typically, cpn60-specific nucleic acids are defined with reference to exons, although introns and regulatory sequences associated with cpn60 coding sequences are also within the scope of the present invention. The term “nucleic acid” as used herein encompasses both RNA and DNA, including genomic DNA. The nucleic acid can be double-stranded or single-stranded. The nucleic acid can contain one or more restriction sites.

Generally, a cpn60-specific nucleic acid marker will be all or a portion of the genomic nucleic acid coding sequence of a cpn60 protein. A cpn60-specific nucleic acid may be specific to a particular species of microbe or may be universal. Species-specific cpn60specific nucleic acid sequences are cpn60 nucleic acid sequences that hybridize preferentially to cpn60 nucleic acid sequences from a given species under appropriate assay conditions. One of skill in the art can design probes to detect such species-specific cpn60-specific nucleic acid sequences by e.g., aligning cpn60 nucleic acid coding sequences and looking for variable regions, e.g., sequences that would not cross-hybridize under the appropriate assay conditions to cpn60 nucleic acid sequences from other species. Alternatively, one of skill in the art will recognize that variable regions, e.g., those that demonstrate no more than 99% sequence similarity (e.g., no more than 85%, 86%, 87%, 88%, 89%, 90%, 91%, 92%, 93%, 94%, 95%, 96%, 97%, and 98% sequence similarity) to a cpn60-specific nucleic acid from another species may also be useful as species-specific cpn60-specific nucleic acids. Use of such specific probes in the methods described herein allows the discriminatory detection of a particular species in a sample.

In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It will be appreciated that a single sequence can align differently with other sequences and hence, can have different percent sequence identity values over each aligned region. It is noted that the percent identity value is usually rounded to the nearest integer. For example, 78.1%, 78.2%, 78.3%, and 78.4% are rounded down to 78%, while 78.5%, 78.6%, 78.7%, 78.8%, and 78.9% are rounded up to 79%. It is also noted that the length of the aligned region is always an integer.

The alignment of two or more sequences to determine percent sequence identity is performed using the algorithm described by Altschul et al. (1997, Nucleic Acids Res., 25:3389-3402) as incorporated into BLAST (basic local alignment search tool) programs, available at http://www.ncbi.nlm.nih.gov. BLAST searches can be performed to determine percent sequence identity between a cpn60-specific nucleic acid sequence from one organism and a cpn60-specific nucleic acid sequence from another organism aligned using the Altschul et al. algorithm. BLASTN is the program used to align and compare the identity between nucleic acid sequences, while BLASTP is the program used to align and compare the identity between amino acid sequences. When utilizing BLAST programs to calculate the percent identity between cpn60 sequences, the default parameters of the respective programs are used.

As used herein, a “universal” cpn60-specific nucleic acid is a cpn60 nucleic acid sequence that is capable of hybridizing under the appropriate assay conditions to one or more cpn60 nucleic acid coding sequences from other microbes. Such sequences, of course, would not hybridize to non-cpn60 nucleic acids under the same assay conditions. One of skill in the art will recognize that hybridization assay conditions can be manipulated in a variety of ways to increase or decrease stringency, e.g., by salt, temperature, choice of buffer, etc. See e.g., Sambrook et al., Molecular Cloning; A Laboratory Manual, 2^{nd} Ed., Cold Spring Harbor Laboratory Press, 1989. Alternatively, one of skill in the art will recognize that nucleic acid sequences demonstrating greater than 75%, 80%, 85%, 90%, or 95% sequence similarity to at least a second cpn60 nucleic acid sequence may be useful as universal cpn60-specific nucleic acids. For example, cpn60 coding sequences from a particular bacterial genera (e.g., Staphylococcus), or sequences derived therefrom, may cross-hybridize under the appropriate assay conditions or have sufficiently similar sequences to one or more cpn60 coding sequences within members of the genus or across genera. As one of skill in the art will recognize, and as explained more fully below, a “universal” probe can then be designed that is capable of detecting two or more of such similar sequences in a sample. For example, one of skill in the art can align cpn60 coding sequences (e.g., from a given genera) and look for sequences that have sequence identity; these sequences thus would be capable of cross-hybridizing to two or more members of the genera. In addition, varying hybridization stringencies can be tested to ascertain optimal conditions for cross-hybridization. Detection of such universal cpn60-specific nucleic acids allows the detection of two or more microbes in a sample, e.g., the detection of all members of a genera, as described previously.

As used herein, a cpn60-specific polypeptide marker is a polypeptide that includes all or a portion of a cpn60 protein. As with cpn60-specific nucleic acids, a cpn60-specific polypeptide marker can be specific to a particular microbial species or universal. A species-specific cpn60-specific polypeptide marker is all or a portion of a given species' cpn60 protein. In the methods of the present invention, the probe or analytical method for detecting the marker should be capable of discriminating between the particular cpn60-specific polypeptide and all other cpn60-specific polypeptides, e.g., by mass in mass-spectrometry applications or by a particular epitope in an antibody assay. For example, and as described more fully below, one of skill in the art will recognize that antibodies, particularly monoclonal antibodies, can be obtained that recognize an epitope that is specific to a particular species' cpn60 protein. Accordingly, use of such specific antibodies in the methods described herein allows the differential detection of a particular species in a sample.

In other embodiments, a cpn60-specific polypeptide marker can be universal. For example, a “universal” cpn60-specific polypeptide marker may be a common structural (conformational) epitope in two or more cpn60 proteins. As described more fully below, antibodies, particularly polyclonal antibodies, raised against cpn60 proteins or polypeptides may be screened for cross-reactivity to common epitopes on cpn60-specific polypeptides from two or more microbes.

Nucleic Acid-Based Assays

Real-Time PCR Assays

Nucleic acid-based methods for identifying and/or quantitating the amount of a microbe in a sample can include amplification of a cpn60 nucleic acid. Amplification methods such as PCR provide powerful means by which to increase the amount of a particular nucleic acid sequence. Nucleic acid hybridization also can be included in determining the presence or absence of a microbe in a sample. Probing amplification products with species-specific hybridization probes is one of the most powerful analytical tools available for profiling. The physical matrix for hybridization can be a nylon membrane (e.g., a macroarray) or a microarray (e.g., a microchip), incorporation of one or more hybridization probes into an amplification reaction (e.g., TaqMan® or Molecular Beacon technology), solution-based methods (e.g., ORIGEN technology), or any one of numerous approaches devised for clinical diagnostics. As discussed above, probes can be designed to preferentially hybridize to amplification products from individual species or to discriminate specific species.

U.S. Pat. Nos. 4,683,202, 4,683,195, 4,800,159, and 4,965,188 disclose conventional PCR techniques. PCR typically employs two oligonucleotide primers that bind to a selected nucleic acid template (e.g., DNA or RNA). Primers useful in the present invention include oligonucleotide primers capable of acting as a point of initiation of nucleic acid synthesis within or adjacent to cpn60 sequences (see below). A primer can be purified from a restriction digest by conventional methods, or can be produced synthetically. Primers typically are single-stranded for maximum efficiency in amplification, but a primer can be double-stranded. Double-stranded primers are first denatured (e.g., treated with heat) to separate the strands before use in amplification. Primers can be designed to amplify a nucleotide sequence from a particular microbial species, or can be designed to amplify a sequence from more than one species. Primers that can be used to amplify a nucleotide sequence from more than one species are referred to herein as “universal primers.” PCR assays can employ template nucleic acids such as DNA or RNA, including messenger RNA (mRNA). The template nucleic acid need not be purified; it can be a minor fraction of a complex mixture, such as a microbial nucleic acid contained in animal cells. Template DNA or RNA can be extracted from a biological or non-biological sample using routine techniques such as those described in Diagnostic Molecular Microbiology: Principles and Applications (Persing et al. (eds.), 1993, American Society for Microbiology, Washington D.C.). Nucleic acids can be obtained from any of a number of sources, including plasmids, bacteria, yeast, organelles, and higher organisms such as plants and animals. Standard conditions for generating a PCR product are well known in the art (see, e.g., PCR Primer: A Laboratory Manual, Dieffenbach and Dveksler (eds.), Cold Spring Harbor Laboratory Press, 1995).

Once a PCR amplification product is generated, it can be detected by, for example, hybridization using FRET technology. FRET technology (see, for example, U.S. Pat. Nos. 4,996,143, 5,565,322, 5,849,489, and 6,162,603) is based on the concept that when a donor fluorescent moiety and a corresponding acceptor fluorescent moiety are positioned within a certain distance of each other, energy transfer taking place between the two fluorescent moieties can be visualized or otherwise detected and quantitated. Two oligonucleotide probes, each containing a fluorescent moiety, can hybridize to an amplification product at particular positions determined by the complementarity of the oligonucleotide probes to the target nucleic acid sequence. Upon hybridization of the oligonucleotide probes to the amplification product at the appropriate positions, a FRET signal is generated. Hybridization temperatures and times can range from about 35° C. to about 65° C. for about 10 seconds to about 1 minute. Detection of FRET can occur in real-time, such that the increase in an amplification product after each cycle of a PCR assay is detected and, in some embodiments, quantified.

Fluorescent analysis and quantification can be carried out using, for example, a photon counting epifluorescent microscope system (containing the appropriate dichroic mirror and filters for monitoring fluorescent emission in a particular range of wavelengths), a photon counting photomultiplier system, or a fluorometer. Excitation to initiate energy transfer can be carried out with an argon ion laser, a high intensity mercury arc lamp, a fiber optic light source, or another high intensity light source appropriately filtered for excitation in the desired range.

Fluorescent moieties can be, for example, a donor moiety and a corresponding acceptor moiety. As used herein with respect to donor and corresponding acceptor fluorescent moieties, “corresponding” refers to an acceptor fluorescent moiety having an emission spectrum that overlaps the excitation spectrum of the donor fluorescent moiety. The wavelength maximum of the emission spectrum of an acceptor fluorescent moiety typically should be at least 100 nm greater than the wavelength maximum of the excitation spectrum of the donor fluorescent moiety, such that efficient non-radiative energy transfer can be produced therebetween.

Fluorescent donor and corresponding acceptor moieties are generally chosen for (a) high efficiency Forster energy transfer; (b) a large final Stokes shift (>100 nm); (c) shift of the emission as far as possible into the red portion of the visible spectrum (>600 nm); and (d) shift of the emission to a higher wavelength than the Raman water fluorescent emission produced by excitation at the donor excitation wavelength. For example, a donor fluorescent moiety can be chosen with an excitation maximum near a laser line (for example, Helium-Cadmium 442 nm or Argon 488 nm), a high extinction coefficient, a high quantum yield, and a good overlap of its fluorescent emission with the excitation spectrum of the corresponding acceptor fluorescent moiety. A corresponding acceptor fluorescent moiety can be chosen that has a high extinction coefficient, a high quantum yield, a good overlap of its excitation with the emission of the donor fluorescent moiety, and emission in the red part of the visible spectrum (>600 nm).

Representative donor fluorescent moieties that can be used with various acceptor fluorescent moieties in FRET technology include fluorescein, Lucifer Yellow, B-phycoerythrin, 9-acridineisothiocyanate, Lucifer Yellow VS, 4-acetamido-4′-isothio-cyanatostilbene-2,2′-disulfonic acid, 7-diethylamino-3-(4′-isothiocyanatophenyl)-4-methylcoumarin, succinimdyl 1-pyrenebutyrate, and 4-acetamido-4′-isothiocyanatostilbene-2,2′-disulfonic acid derivatives. Representative acceptor fluorescent moieties, depending upon the donor fluorescent moiety used, include LC™-Red 640, LC™-Red 705, Cy5, Cy5.5, Lissamine rhodamine B sulfonyl chloride, tetramethyl rhodamine isothiocyanate, rhodamine x isothiocyanate, erythrosine isothiocyanate, fluorescein, diethylenetriamine pentaacetate, and other chelates of Lanthanide ions (e.g., Europium, or Terbium). Donor and acceptor fluorescent moieties can be obtained from, for example, Molecular Probes, Inc. (Eugene, Oreg.) or Sigma Chemical Co. (St. Louis, Mo.).

Donor and acceptor fluorescent moieties can be attached to probe oligonucleotides via linker arms. The length of each linker arm is important, as the linker arms will affect the distance between the donor and acceptor fluorescent moieties. The length of a linker arm for the purpose of the present invention is the distance in Angstroms (Å) from the nucleotide base to the fluorescent moiety. In general, a linker arm is from about 10 to about 25 Å in length. The linker arm may be of the kind described in WO 84/03285, for example. WO 84/03285 also discloses methods for attaching linker arms to a particular nucleotide base, as well as methods for attaching fluorescent moieties to a linker arm.

An acceptor fluorescent moiety such as an LC™-Red 640-NHS-ester can be combined with C6-Phosphoramidites (available from ABI (Foster City, Calif.) or Glen Research (Sterling, Va.)) to produce, for example, LC™-Red 640-Phosphoramidite. Linkers frequently used to couple a donor fluorescent moiety such as fluorescein to an oligonucleotide include thiourea linkers (FITC-derived, for example, fluorescein-CPG's from Glen Research or ChemGene (Ashland, Mass.)), amide-linkers (fluorescein-NHS-ester-derived, such as fluorescein-CPG from BioGenex (San Ramon, Calif.)), or 3‘-amino-CPG’s that require coupling of a fluorescein-NHS-ester after oligonucleotide synthesis.

Using commercially available real-time PCR instrumentation (e.g., LightCycler™, Roche Molecular Biochemicals, Indianapolis, Ind.), PCR amplification, detection, and quantification of an amplification product can be combined in a single closed cuvette with dramatically reduced cycling time. Since detection and quantification occur concurrently with amplification, real-time PCR methods obviate the need for manipulation of the amplification product, and diminish the risk of cross-contamination between amplification products. Real-time PCR greatly reduces turn-around time and is an attractive alternative to conventional PCR techniques in the clinical laboratory, in the field, or at the point of care.

Conventional PCR methods in conjunction with FRET technology can be used to practice the methods of the invention. In one embodiment, a LightCycler™ instrument is used. A detailed description of the LightCycler™ System and real-time and on-line monitoring of PCR can be found at the Roche website. The following patent applications describe real-time PCR as used in the LightCycler™ technology: WO 97/46707, WO 97/46714, and WO 97/46712. The LightCycler™ instrument is a rapid thermal cycler combined with a microvolume fluorometer utilizing high quality optics. This rapid thermocycling technique uses thin glass cuvettes as reaction vessels. Heating and cooling of the reaction chamber are controlled by alternating heated and ambient air. Due to the low mass of air and the high ratio of surface area to volume of the cuvettes, very rapid temperature exchange rates can be achieved within the LightCycler™ thermal chamber. Addition of selected fluorescent dyes to the reaction components allows the PCR to be monitored in real-time and on-line. Furthermore, the cuvettes serve as an optical element for signal collection (similar to glass fiber optics), concentrating the signal at the tip of the cuvette. The effect is efficient illumination and fluorescent monitoring of microvolume samples.

The LightCycler™ carousel that houses the cuvettes can be removed from the instrument. Therefore, samples can be loaded outside of the instrument (in a PCR Clean Room, for example). In addition, this feature allows for the sample carousel to be easily cleaned and sterilized. The fluorometer, as part of the LightCycler™ apparatus, houses the light source. The emitted light is filtered and focused by an epi-illumination lens onto the top of the cuvette. Fluorescent light emitted from the sample is then focused by the same lens, passed through a dichroic mirror, filtered appropriately, and focused onto data-collecting photohybrids. The optical unit currently available in the LightCycler™ instrument (Roche Molecular Biochemicals, Catalog No. 2 011 468) includes three band-pass filters (530 nm, 640 nm, and 710 nm), providing three-color detection and several fluorescence acquisition options. Data collection options include once per cycling step monitoring, fully continuous single-sample acquisition for melting curve analysis, continuous sampling (in which sampling frequency is dependent on sample number) and/or stepwise measurement of all samples after defined temperature interval.

The LightCycler™ can be operated and the data retrieved using a PC workstation and a Windows operating system. Signals from the samples are obtained as the machine positions the capillaries sequentially over the optical unit. The software can display the presence and amount of fluorescent signals in real-time immediately after each measurement. Fluorescent acquisition time is 10-100 milliseconds (msec). After each cycling step, a quantitative display of fluorescence vs. cycle number can be continually updated for all samples. The generated data can be stored for further analysis.

Real-time PCR methods include multiple cycling steps, each step including an amplification step and a hybridization step. In addition, each cycling step typically is followed by a FRET detecting step to detect hybridization of one or more probes to an amplification product. The presence of an amplification product is indicative of the presence of one or more cpn60-containing species. As used herein, “cpn60-containing species” refers to microbes that contain cpn60 nucleic acid sequences. Generally, the presence of FRET indicates the presence of one or more cpn60-containing species in the sample, and the absence of FRET indicates the absence of a cpn60-containing species in the sample. Typically, detection of FRET within, for example, 20, 25, 30, 35, 40, or 45 cycling steps is indicative of the presence of a cpn60-containing species.

As described herein, cpn60 amplification products can be detected using labeled hybridization probes that take advantage of FRET technology. A common format of FRET technology utilizes two hybridization probes that generally are designed to hybridize in close proximity to each other, where one probe is labeled with a donor fluorescent moiety and the other is labeled with a corresponding acceptor fluorescent moiety. Thus, two cpn60 probes can be used, one labeled with a donor fluorophore and the other labeled with a corresponding acceptor fluorophore. The presence of FRET between the donor fluorescent moiety of the first cpn60 probe and the corresponding acceptor fluorescent moiety of the second cpn60 probe is detected upon hybridization of the cpn60 probes to the cpn60 amplification product. For example, a donor fluorescent moiety such as fluorescein is excited at 470 nm by the light source of the LightCycler™ Instrument. During FRET, the fluorescein transfers its energy to an acceptor fluorescent moiety such as LightCycler™-Red 640 (LC™-Red 640) or LightCycler™-Red 705 (LC™-Red 705). The acceptor fluorescent moiety then emits light of a longer wavelength, which is detected by the optical detection system of the LightCycler™ instrument. Efficient FRET can only take place when the fluorescent moieties are in direct local proximity and when the emission spectrum of the donor fluorescent moiety overlaps with the absorption spectrum of the acceptor fluorescent moiety. The intensity of the emitted signal can be correlated with the number of original target DNA molecules (e.g., the number of copies of cpn60).

Another FRET format can include the use of TaqMan® technology to detect the presence or absence of a cpn60 amplification product, and hence, the presence or absence of cpn60-containing species. TaqMan® technology utilizes one single-stranded hybridization probe labeled with two fluorescent moieties. When a first fluorescent moiety is excited with light of a suitable wavelength, the absorbed energy is transferred to a second fluorescent moiety according to the principles of FRET. The second fluorescent moiety generally is a quencher molecule. During the annealing step of the PCR reaction, the labeled hybridization probe binds to the target DNA (i.e., the cpn60 amplification product) and is degraded by the 5′ to 3′ exonuclease activity of the Taq Polymerase during the subsequent elongation phase. As a result, the excited fluorescent moiety and the quencher moiety become spatially separated from one another. As a consequence of excitation of the first fluorescent moiety in the absence of the quencher, the fluorescence emission from the first fluorescent moiety can be detected. By way of example, an ABI PRISM® 7700 Sequence Detection System (Applied Biosystems, Foster City, Calif.) uses TaqMan® technology, and is suitable for performing the methods described herein for detecting cpn60-containing species. Information on PCR amplification and detection using an ABI PRISM® 770 system can be found at the Applied Biosystems website (world wide web at appliedbiosystems.com/products).

Molecular beacons in conjunction with FRET also can be used to detect the presence of a cpn60 amplification product using the real-time PCR methods of the invention. Molecular beacon technology uses a hybridization probe labeled with a first fluorescent moiety and a second fluorescent moiety. The second fluorescent moiety generally is a quencher, and the fluorescent labels typically are located at each end of the probe. Molecular beacon technology uses an oligonucleotide probe having sequences that permit secondary structure formation (e.g., a hairpin). As a result of secondary structure formation within the probe, both fluorescent moieties are in spatial proximity when the probe is in solution. After hybridization to the target cpn60 amplification product, the secondary structure of the probe is disrupted and the fluorescent moieties become separated from one another such that after excitation with light of a suitable wavelength, the emission of the first fluorescent moiety can be detected.

The amount of FRET corresponds to the amount of amplification product, which in turn corresponds to the amount of template nucleic acid present in the sample. Similarly, the amount of template nucleic acid corresponds to the amount of microbial organism present in the sample. Therefore, the amount of FRET produced when amplifying nucleic acid obtained from a biological sample can be correlated to the amount of a microorganism. Typically, the amount of a microorganism in a sample can be quantified by comparing to the amount of FRET produced from amplified nucleic acid obtained from known amounts of the microorganism (e.g., a standard curve). Accurate quantitation requires measuring the amount of FRET while amplification is increasing linearly. In addition, there must be an excess of probe in the reaction. Furthermore, the amount of FRET produced in the known samples used for comparison purposes can be standardized for particular reaction conditions, such that it is not necessary to isolate and amplify samples from every microorganism for comparison purposes.

As an alternative to FRET, a cpn60 amplification product can be detected using, for example, a fluorescent DNA binding dye (e.g., SYBRGreenI® or SYBRGold® (Molecular Probes)). Upon interaction with an amplification product, such DNA binding dyes emit a fluorescent signal after excitation with light at a suitable wavelength. A double-stranded DNA binding dye such as a nucleic acid intercalating dye also can be used. When double-stranded DNA binding dyes are used, a melting curve analysis usually is performed for confirmation of the presence of the amplification product.

Melting curve analysis is an additional step that can be included in a cycling profile. Melting curve analysis is based on the fact that a nucleic acid sequence melts at a characteristic temperature (Tm), which is defined as the temperature at which half of the DNA duplexes have separated into single strands. The melting temperature of a DNA molecule depends primarily upon its nucleotide composition. A DNA molecule rich in G and C nucleotides has a higher Tm than one having an abundance of A and T nucleotides. The temperature at which the FRET signal is lost correlates with the melting temperature of a probe from an amplification product. Similarly, the temperature at which signal is generated correlates with the annealing temperature of a probe with an amplification product. The melting temperature(s) of cpn60 probes from an amplification product can confirm the presence or absence of cpn60-containing species in a sample, and can be used to quantify the amount of a particular cpn60-containing species. For example, a universal probe that hybridizes to a variable region within cpn60 will have a Tm that depends upon the sequence to which it hybridizes. Thus, a universal probe may have a Tm of 70° C. when hybridized to a cpn60 amplification product generated from one microbial organism, but a Tm of 65° C. when hybridized to a cpn60 amplification product generated from a second microbial organism. By observing a temperature-dependent, step-wise decrease in fluorescence of a sample as it is heated, the particular cpn60-containing species in the sample can be identified and the relative amounts of the species in the sample can be determined.

Within each thermocycler run, control samples can be cycled as well. Positive control samples can amplify a nucleic acid control template (e.g., a nucleic acid other than cpn60) using, for example, control primers and control probes. Positive control samples also can amplify, for example, a plasmid construct containing a cpn60 nucleic acid molecule. Such a plasmid control can be amplified internally (e.g., within the sample) or in a separate sample run side-by-side with the test samples. Each thermocycler run also should include a negative control that, for example, lacks cpn60 template DNA. Such controls are indicators of the success or failure of the amplification, hybridization and/or FRET reaction. Therefore, control reactions can readily determine, for example, the ability of primers to anneal with sequence-specificity and to initiate elongation, as well as the ability of probes to hybridize with sequence-specificity and for FRET to occur.

In one embodiment, methods of the invention include steps to avoid contamination. For example, an enzymatic method utilizing uracil-DNA glycosylase is described in U.S. Pat. Nos. 5,035,996, 5,683,896 and 5,945,313, and can be used to reduce or eliminate contamination between one thermocycler run and the next. In addition, standard laboratory containment practices and procedures are desirable when performing methods of the invention. Containment practices and procedures include, but are not limited to, separate work areas for different steps of a method, containment hoods, barrier filter pipette tips and dedicated air displacement pipettes. Consistent containment practices and procedures by personnel are necessary for accuracy in a diagnostic laboratory handling clinical samples.

It is understood that the present invention is not limited by the configuration of one or more commercially available instruments.

Fluorescent In Situ Hybridization (FISH)

In situ hybridization methods such as FISH also can be used to determine a microbial profile. In general, in situ hybridization methods provided herein include the steps of fixing a biological sample, hybridizing a cpn60 probe to target DNA contained within the fixed biological sample, washing to remove non-specific binding, detecting the hybridized probe, and quantifying the amount of hybridized probe.

Typically, cells are harvested from a biological sample using standard techniques. For example, cells can be harvested by centrifuging a biological sample and resuspending the pelleted cells in, for example, phosphate-buffered saline (PBS). After re-centrifuging the cell suspension to obtain a cell pellet, the cells can be fixed in a solution such as an acid alcohol solution, an acid acetone solution, or an aldehyde such as formaldehyde, paraformaldehyde, or glutaraldehyde. For example, a fixative containing methanol and glacial acetic acid in a 3:1 ratio, respectively, can be used as a fixative. A neutral buffered formalin solution also can be used (e.g., a solution containing approximately 1% to 10% of 37-40% formaldehyde in an aqueous solution of sodium phosphate). Slides containing the cells can be prepared by removing a majority of the fixative, leaving the concentrated cells suspended in only a portion of the solution.

The cell suspension is applied to slides such that the cells do not overlap on the slide. Cell density can be measured by a light or phase contrast microscope. For example, cells harvested from a 20 to 100 ml urine sample typically are resuspended in a final volume of about 100 to 200 μl of fixative. Three volumes of this suspension (e.g., 3, 10, and 30 μl), are then dropped into 6 mm wells of a slide. The cellularity (i.e., the density of cells) in these wells is then assessed with a phase contrast microscope. If the well containing the greatest volume of cell suspension does not have enough cells, the cell suspension can be concentrated and placed in another well.

Probes for FISH are chosen for maximal sensitivity and specificity. Using a set of probes (e.g., two or more cpn60 probes) can provide greater sensitivity and specificity than the use of any one probe. Probes typically are about 50 to about 2×10³ nucleotides in length (e.g., 50, 75, 100, 200, 300, 400, 500, 750, 1000, 1500, or 2000 nucleotides in length). Longer probes can comprise smaller fragments of about 100 to about 500 nucleotides in length. Probes that hybridize with locus-specific DNA can be obtained commercially from, for example, Vysis, Inc. (Downers Grove, Ill.), Molecular Probes, Inc. (Eugene, Oreg.), or from Cytocell (Oxfordshire, UK). Alternatively, probes can be made non-commercially from chromosomal or genomic DNA through standard techniques. For example, sources of DNA that can be used include genomic DNA, cloned DNA sequences, somatic cell hybrids that contain one, or a part of one, human chromosome along with the normal chromosome complement of the host, and chromosomes purified by flow cytometry or microdissection. The region of interest can be isolated through cloning, or by site-specific amplification via PCR. See, for example, Nath and Johnson, Biotechnic Histochem., 1998, 73(1):6-22, Wheeless et al., Cytometry, 1994, 17:319-326, and U.S. Pat. No. 5,491,224.

Probes for FISH typically are directly labeled with a fluorescent moiety (also referred to as a fluorophore), an organic molecule that fluoresces after absorbing light of lower wavelength/higher energy. The fluorescent moiety allows the probe to be visualized without a secondary detection molecule. After covalently attaching a fluorophore to a nucleotide, the nucleotide can be directly incorporated into a probe using standard techniques such as nick translation, random priming, and PCR labeling. Alternatively, deoxycytidine nucleotides within a probe can be transaminated with a linker. A fluorophore then can be covalently attached to the transaminated deoxycytidine nucleotides. See, U.S. Pat. No. 5,491,224. The amount of fluorophore incorporated into a probe can be known or determined, and this value in turn can be used to determine the amount of nucleic acid to which the probe binds. In conjunction with analysis of samples (e.g., a serial dilution of a sample) containing known numbers of microbial organisms, the number of microbial organisms in a biological or non-biological sample can be determined.

When more than one probe is used, fluorescent moieties of different colors can be chosen such that each probe in the set can be distinctly visualized and quantitated. For example, a combination of the following fluorophores may be used: 7-amino-4-methylcoumarin-3-acetic acid (AMCA), Texas Red™ (Molecular Probes, Inc.), 5-(and-6)carboxy-X-rhodamine, lissamine rhodamine B, 5-(and-6)-carboxyfluorescein, fluorescein-5-isothiocyanate (FITC), 7-diethylaminocoumarin-3-carboxylic acid, tetramethylrhodamine-5-(and-6)-isothiocyanate, 5-(and-6)-carboxytetramethylrhodamine, 7-hydroxycoumarin-3-carboxylic acid, 6-[fluorescein 5-(and-6)-carboxamido]hexanoic acid, N-(4,4-difluoro-5,7-dimethyl-4-bora-3a,4a diaza-3-indacenepropionic acid, eosin-5-isothiocyanate, erythrosin-5-isothiocyanate, and Cascade™ blue acetylazide (Molecular Probes, Inc.). Probes can be viewed with a fluorescence microscope and an appropriate filter for each fluorophore, or by using dual or triple band-pass filter sets to observe multiple fluorophores. See, for example, U.S. Pat. No. 5,776,688. Alternatively, techniques such as flow cytometry can be used to examine and quantitate the hybridization pattern of the probes.

Probes also can be indirectly labeled with biotin or digoxygenin, or labeled with radioactive isotopes such as ³²P and ³H, although secondary detection molecules or further processing then may be required to visualize the probes and quantify the amount of hybridization. For example, a probe indirectly labeled with biotin can be detected and quantitated using avidin conjugated to a detectable enzymatic marker such as alkaline phosphatase or horseradish peroxidase. Enzymatic markers can be detected and quantitated in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

Prior to in situ hybridization, the probes and the chromosomal DNA contained within the cell each are denatured. Denaturation typically is performed by incubating in the presence of high pH, heat (e.g., temperatures from about 70° C. to about 95° C.), organic solvents such as formamide and tetraalkylammonium halides, or combinations thereof. For example, chromosomal DNA can be denatured by a combination of temperatures above 70° C. (e.g., about 73° C.) and a denaturation buffer containing 70% formamide and 2×SSC (0.3 M sodium chloride and 0.03 M sodium citrate). Denaturation conditions typically are established such that cell morphology is preserved. Probes can be denatured by heat (e.g., by heating to about 73° C. for about five minutes).

After removal of denaturing chemicals or conditions, probes are annealed to the chromosomal DNA under hybridizing conditions. “Hybridizing conditions” are conditions that facilitate annealing between a probe and target chromosomal DNA. Hybridization conditions vary, depending on the concentrations, base compositions, complexities, and lengths of the probes, as well as salt concentrations, temperatures, and length of incubation. The higher the concentration of probe, the higher the probability of forming a hybrid. For example, in situ hybridizations typically are performed in hybridization buffer containing 1-2×SSC, 50% formamide, and blocking DNA to suppress non-specific hybridization. In general, hybridization conditions, as described above, include temperatures of about 25° C. to about 55° C., and incubation times of about 0.5 hours to about 96 hours. More particularly, hybridization can be performed at about 32° C. to about 40° C. for about 2 to about 16 hours.

Non-specific binding of probes to DNA outside of the target region can be removed by a series of washes. The temperature and concentration of salt in each wash depend on the desired stringency. For example, for high stringency conditions, washes can be carried out at about 65° C. to about 80° C., using 0.2× to about 2×SSC, and about 0.1% to about 1% of a non-ionic detergent such as Nonidet P-40 (NP40). Stringency can be lowered by decreasing the temperature of the washes or by increasing the concentration of salt in the washes.

mRNA-Based Assays

Alternatively, in order to test for the presence or absence of, or measure the level of, a cpn60-specific mRNA in a sample, e.g., a sample comprising cells, the cells can be lysed and total RNA can be purified or semi-purified from lysates by any of a variety of methods known in the art. Methods of detecting or measuring levels of particular mRNA transcripts are also familiar to those in the art. Such assays include, without limitation, hybridization assays using detectably labeled cpn60-specific nucleic acid (DNA or RNA) probes and quantitative or semi-quantitative RT-PCR methodologies employing appropriate cpn60 oligonucleotide primers. Additional methods for quantitating mRNA in cell lysates include RNA protection assays and serial analysis of gene expression (SAGE). Alternatively, qualitative, quantitative, or semi-quantitative in situ hybridization assays can be carried out using, for example, samples such as tissue sections or unlysed cell suspensions, and detectably (e.g., fluorescently, isotopically, or enzymatically) labeled DNA or RNA probes.

Polypeptide-Based Assays

The invention also features polypeptide-based assays. A cpn60 protein, or cpn60-specific polypeptide, can be used as a universal target to determine the presence or absence of one or more microbes, and further used as species-specific targets and/or probes for the identification and classification of specific microbes. Such assays can be used on their own or in conjunction with other procedures (e.g., nucleic acid-based assays) to monitor high-risk environments.

In the assays of the invention, the presence or absence of a cpn60-specific polypeptide is detected and/or its level is measured. The presence of a cpn60-specific polypeptide may be measured in a liquid sample such as a body fluid (e.g., urine, milk, lachrymal fluid, vitreous fluid, sputum, cerebrospinal fluid, sweat, lymph, saliva, semen, blood, or serum or plasma derived from blood); a lavage such as a breast duct lavage, lung lavage, a gastric lavage, a rectal or colonic lavage, or a vaginal lavage; an aspirate such as a nipple or teat aspirate; a fluid such as a cell culture or a supernatant from a cell culture; a fluid such as a buffer that has been used to obtain a sample from e.g., a fomite, such as a buffer used to wash or to wet a swab in a swab sampling procedure; and a water sample. In addition, any sample that can be solubilized may also be used in the methods of the present invention.

Methods of detecting or measuring the levels of a protein of interest (e.g., a cpn60 protein, or cpn60-specific polypeptides) in cells are known in the art. Many such methods employ antibodies (e.g., polyclonal antibodies or mAbs) that bind specifically to the protein.

Antibodies and Antibody-Based Assays

Antibodies having specific binding affinities for a cpn60 protein or a cpn60-specific polypeptide may be produced through standard methods. As used herein, the terms “antibody” or “antibodies” include intact molecules as well as fragments thereof which are capable of binding to an epitopic determinant of a cpn60-specific polypeptide. The term “epitope” refers to an antigenic determinant on an antigen to which the paratope of an antibody binds. Epitopic determinants usually consist of chemically active surface groupings of molecules such as amino acids or sugar side chains, and typically have specific three-dimensional structural characteristics, as well as specific charge characteristics. Epitopes generally have at least five contiguous amino acids (a continuous epitope), or alternatively can be a set of noncontiguous amino acids that define a particular structure (e.g., a conformational epitope). The terms “antibody” and “antibodies” include polyclonal antibodies, monoclonal antibodies, humanized or chimeric antibodies, single chain Fv antibody fragments, Fab fragments, and F(ab)₂ fragments.

Antibodies may be specific for a particular cpn60-specific polypeptide, e.g., the cpn60 protein of the Clostridium perfringens microbe. Alternatively, they may be cross-reactive with two or more cpn60-specific polypeptides, e.g., cross-react or bind to two or more cpn60 proteins. For example, such antibodies may bind to common epitopes present in two or more cpn60 proteins or cpn60-specific polypeptides. As used herein, such antibodies with specificity for two or more cpn60-specific polypeptides are termed “universal” antibodies. For example, certain antibodies may bind to common epitopes present in all cpn60-specific polypeptides. Certain of such antibodies thus may be termed able to detect the presence or absence of any microbe in a sample.

In certain embodiments of the method described herein, depending on the high-risk environment and the purpose for monitoring, it may be sufficient to determine simply whether or not any microbe is present, and optionally the relative concentration or amount of the microbe. Such a detection may occur through, e.g., the use of one or more “universal” cpn60 antibodies, such as an antibody that binds or demonstrates specificity to two or more cpn60-specific polypeptides (e.g., one that is cross-reactive with all cpn60 proteins of a particular genera, or with all bacterial cpn60 proteins) as described previously.

In other embodiments, the identification of the particular microbe may be preferred. Accordingly, an antibody specific for a particular cpn60-specific polypeptide may be employed, either alone or in conjunction with a universal antibody; such antibodies are referred to as “specific” antibodies herein. The universal and specific antibodies may be employed simultaneously or in series. For example, a universal antibody may be used as a first screen to determine the presence or absence of a cpn60-specific polypeptide. Subsequently, a specific antibody, such as one specific for a cpn60-specific polypeptide of a particular microbe, e.g., Campylobacter jejuni, may be employed. In such assays, monoclonal antibodies may be particularly useful (e.g., sensitive) to identify cpn60-specific polypeptides of a particular microbe.

In general, a protein of interest (e.g., a cpn60 protein against which one wishes to prepare antibodies) is produced recombinantly, by chemical synthesis, or by purification of the native protein, and then used to immunize animals. As used herein, an intact cpn60 protein may be employed, or a cpn60-specific polypeptide may be employed, provided that the cpn60-specific polypeptide is capable of generating the desired immune response. See, for example, WO 200265129 for examples of epitopic sequences that bind to human antibodies against Chlamydia trachomatis; such epitopic sequences may be useful in generating antibodies against Chlamydia spp. for use in the present invention. See also U.S. Pat. No. 6,497,880, which sets forth nucleic acid sequences, amino acid sequences, expression vectors, purified proteins, antibodies, etc. specific to Aspergillus fumigatus and Candida glabrata. Purified Aspergillus fumigatus and Candida glabrata cpn60 proteins, or proteolytically or synthetically generated fragments thereof, can be used to immunize animals to generate antibodies for use in the methods of the present invention. Finally, see WO 200257784, disclosing substantially purified Chlamydia hsp60 (cpn60) polypeptides. Such polypeptides may also be used to generate antibodies for use in the methods of the present invention.

As discussed previously, one may wish to prepare universal or specific antibodies to cpn60 proteins or polypeptides. A cpn60-specific polypeptide may be used to generate a universal antibody, for example, if it maintains an epitope that is common to at least two cpn60 proteins, or, e.g., to all cpn60 proteins that one wishes to detect (e.g., the cpn60 proteins of the Campylobacter genera). Alternatively, a cpn60 protein or cpn60-specific polypeptide may be used to generate antibodies specific for a particular cpn60 protein or polypeptide present in a particular microbe, e.g., only Campylobacter jejuni.

Various host animals including, for example, rabbits, chickens, mice, guinea pigs, and rats, can be immunized by injection of the protein of interest. Adjuvants can be used to increase the immunological response depending on the host species and include Freund's adjuvant (complete and incomplete), mineral gels such as aluminum hydroxide, surface active substances such as lysolecithin, pluronic polyols, polyanions, peptides, oil emulsions, keyhole limpet hemocyanin (KLH), and dinitrophenol. Polyclonal antibodies are heterogenous populations of antibody molecules that are specific for a particular antigen, which are contained in the sera of the immunized animals. Monoclonal antibodies, which are homogeneous populations of antibodies to a particular epitope contained within an antigen, can be prepared using standard hybridoma technology. In particular, monoclonal antibodies can be obtained by any technique that provides for the production of antibody molecules by continuous cell lines in culture such as described by Kohler, G. et al., Nature, 1975, 256:495, the human B-cell hybridoma technique (Kosbor et al., Immunology Today, 1983, 4:72; Cole et al., Proc. Natl. Acad. Sci. USA, 1983, 80:2026), and the EBV-hybridoma technique (Cole et al., “Monoclonal Antibodies and Cancer Therapy”, Alan R. Liss, Inc., 1983, pp. 77-96). Such antibodies can be of any immunoglobulin class including IgG, IgM, IgE, IgA, IgD, and any subclass thereof. The hybridoma producing the monoclonal antibodies of the invention can be cultivated in vitro or in vivo.

A chimeric antibody is a molecule in which different portions are derived from different animal species, such as those having a variable region derived from a murine monoclonal antibody and a human immunoglobulin constant region. Chimeric antibodies can be produced through standard techniques.

Antibody fragments that have specific binding affinity for a cpn60-specific polypeptide can be generated by known techniques. For example, such fragments include, but are not limited to, F(ab′)₂ fragments that can be produced by pepsin digestion of the antibody molecule, and Fab fragments that can be generated by reducing the disulfide bridges of F(ab′)₂ fragments. Alternatively, Fab expression libraries can be constructed. See, for example, Huse et al., 1989, Science, 246:1275. Single chain Fv antibody fragments are formed by linking the heavy and light chain fragments of the Fv region via an amino acid bridge (e.g., 15 to 18 amino acids), resulting in a single chain polypeptide. Single chain Fv antibody fragments can be produced through standard techniques. See, for example, U.S. Pat. No. 4,946,778.

Once produced, antibodies or fragments thereof are tested for recognition of a cpn60 protein or cpn60-specific polypeptide by standard immunoassay methods including, for example, ELISA techniques, countercurrent immuno-electrophoresis (CIEP), radioimmunassays (RIA), radioimmunoprecipitations, dot blots, inhibition or competition assays, sandwich assays, immunostick (dipstick) assays, immunochromatographic assays, immunofiltration assays, latex beat agglutination assays, immunofluoroescent assays, biosensor assays. See, Short Protocols in Molecular Biology, Chapter 11, Green Publishing Associates and John Wiley & Sons, Edited by Ausubel, F. M et al., 1992; Antibodies: A Laboratory Manual, Harlow and Lane (eds.), Cold Spring Harbor Laboratory Press, 1988; and U.S. Pat. Nos. 4,376,110; 4,486,530; and 6,497,880. Antibodies or fragments can also be tested for their ability to react universally, e.g., with two or more cpn60 proteins or cpn60-specific polypeptides, such as a subset of cpn60 proteins and polypeptides (e.g., the cpn60 proteins from a bacterial genera such as Clostridium), or specifically with a particular cpn60 protein (e.g., the cpn60 protein of Clostridium perfringens).

In antibody assays, the antibody itself or a secondary antibody that binds to it can be detectably labeled. Alternatively, the antibody can be conjugated with biotin, and detectably labeled avidin (a protein that binds to biotin) can be used to detect the presence of the biotinylated antibody. Combinations of these approaches (including “multi-layer” assays) familiar to those in the art can be used to enhance the sensitivity of assays. Some of these assays (e.g., immunohistological methods or fluorescence flow cytometry) can be applied to histological sections or unlysed cell suspensions. The methods described below for detecting a cpn60-specific polypeptide in a liquid sample can also be used to detect a cpn60-specific polypeptide in cell lysates.

Methods of detecting a cpn60-specific polypeptide in a liquid sample generally involve contacting a sample of interest with an antibody that binds to a cpn60-specific polypeptide and testing for binding of the antibody to a component of the sample. In such assays the antibody need not be detectably labeled and can be used without a second antibody that binds to a cpn60-specific polypeptide. For example, an antibody specific for a cpn60-specific polypeptide may be bound to an appropriate solid substrate and then exposed to the sample. Binding of a cpn60-specific polypeptide to the antibody on the solid substrate may be detected by exploiting the phenomenon of surface plasmon resonance, which results in a change in the intensity of surface plasmon resonance upon binding that can be detected qualitatively or quantitatively by an appropriate instrument, e.g., a Biacore apparatus (Biacore International AB, Rapsgatan, Sweden).

Moreover, assays for detection of a cpn60-specific polypeptide in a liquid sample can involve the use, for example, of: (a) a single antibody specific for a cpn60-specific polypeptide that is detectably labeled; (b) an unlabeled antibody that is specific for a cpn60-specific polypeptide and a detectably labeled secondary antibody; or (c) a biotinylated antibody specific for a cpn60-specific polypeptide and detectably labeled avidin. In addition, combinations of these approaches (including “multi-layer” assays) familiar to those in the art can be used to enhance the sensitivity of assays. In these assays, the sample or an aliquot of the sample suspected of containing a microbe can be immobilized on a solid substrate, such as a nylon or nitrocellulose membrane, by, for example, “spotting” an aliquot of the liquid sample or by blotting of an electrophoretic gel on which the sample or an aliquot of the sample has been subjected to electrophoretic separation. The presence or amount of cpn60-specific polypeptide on the solid substrate is then assayed using any of the above-described forms of the cpn60-specific polypeptide specific antibody and, where required, appropriate detectably labeled secondary antibodies or avidin.

The invention also features “sandwich” assays. In these sandwich assays, instead of immobilizing samples on solid substrates by the methods described above, any cpn60-specific polypeptide that may be present in a sample can be immobilized on the solid substrate by, prior to exposing the solid substrate to the sample, conjugating a second (“capture”) antibody (polyclonal or mAb) specific for a cpn60-specific polypeptide to the solid substrate by any of a variety of methods known in the art. In exposing the sample to the solid substrate with the second antibody specific for cpn60-specific polypeptide bound to it, any cpn60-specific polypeptide in the sample (or sample aliquot) will bind to the second antibody on the solid substrate. The presence or amount of cpn60-specific polypeptide bound to the conjugated second antibody is then assayed using a “detection” antibody specific for a cpn60-specific polypeptide by methods essentially the same as those described above using a single antibody specific for a cpn60-specific polypeptide. It is understood that in these sandwich assays, the capture antibody should not bind to the same epitope (or range of epitopes in the case of a polyclonal antibody) as the detection antibody. Thus, if a mAb is used as a capture antibody, the detection antibody can be either: (a) another mAb that binds to an epitope that is either completely physically separated from or only partially overlaps with the epitope to which the capture mAb binds; or (b) a polyclonal antibody that binds to epitopes other than or in addition to that to which the capture mAb binds. On the other hand, if a polyclonal antibody is used as a capture antibody, the detection antibody can be either (a) a mAb that binds to an epitope to that is either completely physically separated from or partially overlaps with any of the epitopes to which the capture polyclonal antibody binds; or (b) a polyclonal antibody that binds to epitopes other than or in addition to that to which the capture polyclonal antibody binds. Assays that involve the used of a capture and detection antibody include sandwich ELISA assays, sandwich Western blotting assays, and sandwich immunomagnetic detection assays.

Suitable solid substrates to which the capture antibody can be bound include, without limitation, the plastic bottoms and sides of wells of microtiter plates, membranes such as nylon or nitrocellulose membranes, and polymeric (e.g., without limitation, agarose, cellulose, or polyacrylamide) beads or particles. It is noted that antibodies bound to such beads or particles can also be used for immunoaffinity purification of cpn60-specific polypeptides. Dipstick/immunostick formats can employ a solid phase, e.g., polystyrene, paddle or dispstick.

Methods of detecting or for quantifying a detectable label depend on the nature of the label and are known in the art. Appropriate labels include, without limitation, radionuclides (e.g., ¹²⁵I, ¹³¹T, ³⁵S, ³H, ³²P, ³³P, or ¹⁴C), fluorescent moieties (e.g., fluorescein, rhodamine, or phycoerythrin), luminescent moieties (e.g., Qdot™ nanoparticles supplied by the Quantum Dot Corporation, Palo Alto, Calif.), compounds that absorb light of a defined wavelength, or enzymes (e.g., alkaline phosphatase or horseradish peroxidase). The products of reactions catalyzed by appropriate enzymes can be, without limitation, fluorescent, luminescent, or radioactive, or they may absorb visible or ultraviolet light. Examples of detectors include, without limitation, x-ray film, radioactivity counters, scintillation counters, spectrophotometers, calorimeters, fluorometers, luminometers, and densitometers.

The methods of the present invention may employ a control sample. In assays to detect the presence or absence of a microbe, the concentration of a cpn60-specific polypeptide in, for example, a food sample suspected of being contaminated, or at risk of being contaminated, with a microbe may be compared to a control sample, e.g., a food sample known not to be infected. The control sample may be taken from the same high-risk environment, e.g., in a different location known to be uncontaminated, or can be a control sample taken from a non-high-risk environment. Alternatively, the control sample may be taken from the same location of a high-risk environment but at an earlier or later time-point when the location was known to be uncontaminated. A significantly higher concentration of cpn60-specific polypeptide in the suspect sample relative to the control sample would indicate the presence of a microbe.

It is understood that, while the above descriptions of the diagnostic assays may refer to assays on food samples or bodily fluid samples, the assays can also be carried out on any of the other fluid or solubilized samples listed herein, such as water samples or buffer samples (e.g., buffer used to extract a sample from a fomite).

Other Polypeptide Detection Assays

The present invention also contemplates the use of other analytical techniques for detecting cpn60-specific polypeptides. Recent analytical instrumentation and methodology advances that have arisen in the context of proteomics research are applicable in the methods of the present invention. See, generally, P R Jungblut, “Proteome Analysis of Bacterial Pathogens,” Microbes & Infection 3 (2001): 831-840; G MacBeath and S L Schreiber, “Printing Proteins as Microarrays for High-Throughput Function Determination,” Science 289 (2000): 1760-1763; J Madoz-Gdrpide, H Wang, and D E Misek, “Protein-Based Microarrays: A Tool for Probing the Proteome of Cancer Cells and Tissues,” Proteomics 1 (2001): 1279-1287; S Patterson, “Mass Spectrometry and Proteomics,” Physiological Genomics 2 (2000): 59-65; and A Schevchenko et al., “Maldi Quadrupole Time-of-Flight Mass Spectrometry: A Powerful Tool for Proteomic Research,” Analytical Chemistry 72 (2000):2132-2141.

Mass-spectrophotometric techniques have been increasingly used to detect and identify proteins and protein fragments at low levels, e.g., fmol or pmol. Mass spectrometry has become a major analytical tool for protein and proteomics research because of advancements in the instrumentation used for biomolecular ionization, electrospray ionization (ESI), and matrix-assisted laser desorption-ionization (MALDI). MALDI is usually combined with a time-of-flight (TOF) mass analyzer. Typically, 0.5 μl of sample that contains 1-10 pmol of protein or peptide is mixed with an equal volume of a saturated matrix solution and allowed to dry, resulting in the co-crystallization of the analyte with the matrix. Matrix compounds that are used include sinapic acid and α-hydroxycinnamic acid. The cocrystallized material on the target plate is irradiated with a nitrogen laser pulse, e.g., at a wavelength of 337 nm, to volatilize and ionize the protein or peptide molecules. A strong acceleration field is switched on, and the ionized molecules move down the flight tube to a detector. The amount of time required to reach the detector is related to the mass-to-charge ratio. Proteolytic mass mapping and tandem mass spectrometry, when combined with searches of protein and protein fragment databases, can also be employed to detect and identify cpn60-specific polypeptides. See, for example, Devin M. Downard, “Contributions of mass spectrometry to structural immunology,” J. Mass. Spectrom. 35:493-503(2000).

Biomolecular interaction analysis mass spectrometry (BIA-MS) is another suitable technique for detecting interactions between cpn60-specific polynucleotides and cpn60 antibodies. This technology detects molecules bound to a ligand that is covalently attached to a surface. As the density of biomaterial on the surface increases, changes occur in the refractive index at the solution or surface interface. This change in the refractive index is detected by varying the angle or wavelength at which the incident light is absorbed at the surface. The difference in the angle or wavelength is proportional to the amount of material bound on the surface, giving rise to a signal that is termed surface plasmon resonance (SPR), as discussed previously. See, for example, R W Nelson et al., “BIA/MS of Epitope-Tagged Peptides Directly from E. coli Lysate: Multiplex Detection and Protein Identification at Low-Femtomole to Subfemtomolc Levels,” Analytical Chemistry 71 (1999): 2858-2865; see also D Nedelkov and R W Nelson, “Analysis of Native Proteins from Biological Fluids by Biomolecular Interaction Analysis Mass Spectrometry (BIA/MS): Exploring the Limit of Detection, Identification of Non-Specific Binding and Detection of Multiprotein Complexes,” Biosensors and Bioelectronics 16 (2001): 1071-1078.

The SPR biosensing technology has been combined with MALDI-TOF mass spectrometry for the desorption and identification of biomolecules. In a chip-based approach to BIA-MS, a ligand, e.g., a cpn60 antibody, is covalently immobilized on the surface of a chip. A tryptic digest of solubilized proteins from a sample is routed over the chip, and the relevant peptides, e.g., cpn60-specific polypeptides, are bound by the ligand. After a washing step, the eluted peptides are analyzed by MALDI-TOF mass spectrometry. The system may be a fully automated process and is applicable to detecting and characterizing proteins present in complex biological fluids and cell extracts at low- to subfemtomol levels.

Mass spectrometers useful for such applications are available from Applied Biosystems (Foster City, Calif.); Bruker Daltronics (Billerica, Mass.) and Amersham Pharmacia (Sunnyvale, Calif.).

Other suitable techniques for use in the present invention include “Multidimensional Protein Identification Technologies.”. Cells are fractionally solubilized and digested, e.g., sequentially with endoproteinase Lys-C and immobilized trypsin. The samples are then subjected to multidimensional protein identification technology (MUDPIT), which involves a sequential separation of the peptide fragments by on-line biphasic microcapillary chromatography (e.g., strong ion exchange, then C-18 separation), followed by tandem mass spectrometry (MS-MS). See, for example, M P Washburn, D Wolter, and J R Yates, “Large-Scale Analysis of the Yeast Proteome by Multidimensional Protein Identification Technology,” Nature Biotechnology 19 (2001): 242-247.

Articles of Manufacture

The invention also provides articles of manufacture. Articles of manufacture can include at least one cpn60 oligonucleotide primer, as well as instructions for using the cpn60 oligonucleotide(s) to identify and quantify the amount of one or more microbial organisms in a biological or non-biological sample.

In one embodiment, the cpn60 oligonucleotide(s) are attached to a microarray (e.g., a GeneChip®, Affymetrix, Santa Clara, Calif.). In another embodiment, an article of manufacture can include one or more cpn60 oligonucleotide primers and one or more cpn60 oligonucleotide probes. Such cpn60 primers and probes can be used, for example, in real-time amplification reactions to amplify and simultaneously detect cpn60 amplification products.

Suitable oligonucleotide primers include those that are complementary to highly conserved regions of cpn60 and that flank a variable region. Such universal cpn60 primers can be used to specifically amplify these variable regions, thereby providing a sequence with which to identify microorganisms. Examples of cpn60 oligonucleotide primers include the following:

| | |
|---|---|
| 5′-GAIIIIGCIGGIGA(T/C)GGIACIACIAC-3′; and | (SEQ ID NO:6) |
| 5′-(T/C)(T/G)I(T/C)(T/G)ITCICC(A/G)AAICCIGGIGC(T/C)TT-3′. | (SEQ ID NO:7) |

Suitable oligonucleotide primers also include those that are complementary to species-specific cpn60 sequences, and thus result in an amplification product only if a particular species is present in the sample.

Similar to cpn60 oligonucleotide primers, cpn60 oligonucleotide probes generally are complementary to cpn60 sequences. cpn60 oligonucleotide probes can be designed to hybridize universally to cpn60 sequences, or can be designed for species-specific hybridization to the variable region of cpn60 sequences.

An article of manufacture of the invention can further include additional components for carrying out amplification reactions and/or reactions, for example, on a microarray. Articles of manufacture for use with PCR reactions can include nucleotide triphosphates, an appropriate buffer, and a polymerase. An article of manufacture of the invention also can include appropriate reagents for detecting amplification products. For example, an article of manufacture can include one or more restriction enzymes for distinguishing amplification products from different species of microorganism, or can include fluorophore-labeled oligonucleotide probes for real-time detection of amplification products.

It will be appreciated by those of ordinary skill in the art that different articles of manufacture can be provided to evaluate microbes in different types of high-risk environments. For example, a hospital will have a different community of microbes than that of a restaurant. Therefore, an article of manufacture designed to evaluate the microbes in a hospital may have a different set of controls or a different set of species-specific hybridization probes than that designed for a restaurant. Alternatively, a more generalized article of manufacture can be used to evaluate the microbes in a number of different high-risk environments.

Articles of manufacture also can include at least one cpn60 antibody, as well as instructions for using the same to detect the presence of a microbe, and optionally to evaluate a microbial profile, in a biological or non-biological sample.

In one embodiment, one or more cpn60 antibodies are attached to a microarray (e.g., a 96-microwell plate). For example, a microarray format may include a variety of universal and specific cpn60 capture antibodies; the universal and specific antibodies may each be located at a different well location. The article of manufacture may also include the appropriate detection antibodies, if necessary, and appropriate reagents for detection of binding of a cpn60-specific polypeptide to one or more capture antibodies (e.g., enzymes, substrates, buffers, and controls).

In another embodiment, an article of manufacture can include one or more cpn60 antibodies attached to a dipstick. Such dipsticks can be used, for example, to detect cpn60-specific polypeptides in a liquid sample.

### EXAMPLES

Example 1

### Quantitating Microbial Organisms Using Universal Primers and a Universal Probe

A biological sample is obtained from poultry GIT and genomic DNA is extracted using standard methods (Diagnostic Molecular Microbiology: Principles and Applications (supra)). Real-time PCR is conducted using universal cpn60 primers having the nucleotide sequences set forth in SEQ ID NO:6 and SEQ ID NO:7, and a universal cpn60 probe having the sequence 5′-GACAAAGTCGGTAAAGAAGGCGTTATCA-3′ (SEQ ID NO:8), labeled at the 5′ end with fluorescein (fluorophore; Molecular Probes, Inc.) and at the 3′ end with dabcyl (quencher; (4-(4′-dimethylaminophenylazo)benzoic acid) succinimidyl ester; Molecular Probes, Inc.). This probe binds to a variable region of the cpn60 gene from numerous microbial species; thus the Tm of the probe from an amplification product varies depending upon the nucleotide sequence within the amplification product to which the probe hybridizes.

The PCR reaction contains 3 μL extracted DNA, 1 μM each universal cpn60 primer, 340 nM universal cpn60 probe, 2.5 units Amplitaq Gold DNA polymerase (Perkin Elmer), 0.25 mM each deoxyribonucleotide, 3.5 mM MgCl₂, 50 mM KCl, and 10 mM Tris-HCl, pH 8.0 in a total reaction volume of 50 μL. PCR conditions include an initial incubation at 95° C. for 10 minutes to activate the Amplitaq Gold DNA polymerase, followed by 40 cycles of 30 seconds at 95° C., 60 seconds at 50° C., and 30 seconds at 72° C. Fluorescence is monitored during the 50° C. annealing steps throughout the 40 cycles. After the cycling steps are complete, the melting temperature of the universal probe from the amplification products is analyzed. As the temperature is increased, the universal probe is released from the amplification product from each species' cpn60 sequence at a specific temperature, corresponding to the Tm of the universal probe and the cpn60 sequence of the particular species. The step-wise loss of fluorescence at particular temperatures is used to identify the particular species present, and the loss in fluorescence of each step compared to the total amount of fluorescence correlates with the relative amount of each microorganism.

### Example 2

### Quantification of Microbial Organisms Using Universal Primers and Species-Specific Probes

A biological sample is obtained from poultry GIT and genomic DNA is extracted using standard methods (Diagnostic Molecular Microbiology: Principles and Applications (supra)). Real-time PCR is conducted using universal cpn60 primers having the nucleotide sequences set forth in SEQ ID NO:6 and SEQ ID NO:7, and species-specific probes having the nucleotide sequences:

| | |
|---|---|
| 5′-AGCCGTTGCAAAAGCAGGCAAACCGC-3′; | (SEQ ID NO:9) |
| 5′-TTGAGCAAATAGTTCAAGCAGGTAA-3′; | (SEQ ID NO:10) |
| 5′-GCAACTCTGGTTGTTAACACCATGC-3′; | (SEQ ID NO:11) |
| 5′-TGGAGAAGGTCATCCAGGCCAACGC-3′; and | (SEQ ID NO:12) |
| 5′-TAGAACAAATTCAAAAAACAGGCAA-3′. | (SEQ ID NO:13) |

These species-specific probes hybridize to cpn60 nucleotide sequences from S. enterica, C. perfringens, E. coli, S. coelicolor, and C. jejuni, respectively. The sequences of the probes are identified by aligning cpn60 cDNA sequences from the five organisms and identifying a sequence that is specific to each particular organism (i.e., a sequence not found in the other organisms). Each of the species-specific probes is labeled with a different fluorescent moiety to allow differential detection of the various species.

The PCR reaction contains 3 μL extracted DNA, 1 μM each universal cpn60 primer, 340 nM universal cpn60 probe, 2.5 units Amplitaq Gold DNA polymerase (Perkin Elmer), 0.25 mM each deoxyribonucleotide, 3.5 mM MgCl₂, 50 mM KCl, and 10 mM Tris-HCl, pH 8.0 in a total reaction volume of 50 μL. PCR conditions include an initial incubation at 95° C. for 10 minutes to activate the Amplitaq Gold DNA polymerase, followed by 40 cycles of 30 seconds at 95° C., 60 seconds at 50° C., and 30 seconds at 72° C. Fluorescence is monitored during the 50° C. annealing steps throughout the 40 cycles, at wavelengths corresponding to the particular moieties on the probes. The amount of fluorescence detected at each of the monitored wavelengths correlates with the amount of each cpn60 amplification product. The amount of each species-specific amplification product is then correlated with the amount of each species of microbe by comparison to the amount of amplification product generated from samples containing nucleic acid isolated from known amounts of each microbial species.

### Example 3

### Dipstick ELISA Assay for Streptococcus

A polystyrene dipstick containing two horizontal bands is constructed: one band consists of broadly reactive, polyclonal capture antibodies against cpn60 proteins from Streptococcus spp., while the other band is an internal control consisting of horseradish peroxidase. The assay is performed by making serial dilutions (1:2, 1:5, 1:10, etc.) of a liquid sample taken from a high risk environment (e.g., a urine sample or a blood sample) directly into a detection reagent and incubating a wetted dipstick in these dilutions for 5 minutes, and then adding an indicator to detect binding of cpn60 proteins to the capture (and detection) antibodies. The detection reagent includes a suitable buffer and secondary cpn60 Streptococcus detection antibodies labeled with horseradish peroxidase. The indicator can be a chromogenic horseradish peroxidase substrate, such as 2,2′-AZINO-bis 3-ethylbenziazoline-6-sulfonic acid, or ABTS. ABTS is considered a safe, sensitive substrate for horseradish peroxidase that produces a blue-green color upon enzymatic activity that can be quantitated at 405-410 nm. At the end of the incubation and indicator steps, the dipstick is rinsed with water (e.g., deionized water) and examined for staining of the antibody band by visual inspection. Staining of the antibody band reveals the presence of Streptococcus spp. in the sample. The internal control band provides a check on the integrity of the detection reagent.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for monitoring a high-risk environment for the presence or absence of one or more microbes, said method comprising:
a) providing a sample obtained from said high-risk environment; and
b) detecting the presence or absence of a cpn60 marker in said sample, said cpn60 marker indicative of the presence of said one or more microbes.

2. The method of claim 1, wherein said detecting step is capable of providing a microbial profile of said sample.

3. The method of claim 2, wherein said microbial profile comprises an identification of said one or more microbes in said sample.

4. The method of claim 2, wherein said microbial profile comprises a quantification of the amount of said one or more microbes in said sample.

5. The method according to claim 2, further comprising acquiring a microbial profile of said high-risk environment at two or more points.

6. The method according to claim 5, wherein said two or more points are time points.

7. The method according to claim 5, wherein said two or more points are location points.

8. The method according to claim 5, further comprising comparing said microbial profiles at said two or more points.

9. The method according to claim 2, further comprising acquiring a control microbial profile from a control sample from said high-risk environment.

10. The method according to claim 9, further comprising comparing said control sample microbial profile with said sample microbial profile.

11. The method according to claim 1, wherein said cpn60 marker is a cpn60-specific nucleic acid or a cpn60-specific polypeptide.

12. The method according to claim 11, wherein said cpn60-specific nucleic acid is a genomic nucleic acid coding sequence of a cpn60 protein.

13. The method according to claim 12, wherein said genomic nucleic acid coding sequence is of chromosomal origin.

14. The method according to claim 11, wherein said cpn60-specific nucleic acid is an amplified sequence of a cpn60 coding sequence of said microbe.

15. The method according to claim 1, wherein said detecting step is a nucleic acid-based assay.

16. The method according to claim 15, wherein said nucleic acid-based assay is selected from the group consisting of PCR and FISH assays.

17. The method according to claim 1, wherein said detecting step is a polypeptide-based assay.

18. The method according to claim 17, wherein said polypeptide-based assay is an immunodiagnostic assay.

19. The method according to claim 18, wherein said immunodiagnostic assay is ELISA.

20. The method according to claim 17, wherein said polypeptide-based assay comprises a mass spectrometric technique.

21. The method according to claim 17, wherein said polypeptide-based assay comprises a surface plasmon resonance technique.

22. The method according to claim 1, wherein said microbe is selected from the group consisting of bacteria, protozoa, rickettsiae, and fungi.

23. The method according to claim 22, wherein said bacterial microbe is selected from the group consisting of the Staphylococcus, Streptococcus, Pseudomonas, Escherichia, Bacillus, Brucella, Chlamydia, Clostridium, Shigella, Mycobacterium, Agrobacterium, Bartonella, Borellia, Bradyrhizobium, Ehrlichia, Haemophilus, Helicobacter, Heliobacter, Lactobacillus, Neisseria, Rhizobium, Streptomyces, Synechococcus, Zymomonas, Synechocyotis, Mycoplasma, Yersinia, Vibrio, Burkholderia, Franciscella, Legionella, Salmonella, Bifidobacterium, Enterococcus, Enterobacter, Citrobacter, Bacteroides, Prevotella, Xanthomonas, Xylella, and Campylobacter genera.

24. The method according to claim 22, wherein said protozoan microbe is selected from the group consisting of the Acanthamoeba, Cryptosporidium, and Tetrahymena genera.

25. The method according to claim 22, wherein said fungal microbe is selected from the group consisting of Aspergillus, Colletrotrichum, Cochliobolus, Helminthosporium, Microcyclus, Puccinia, Pyricularia, Deuterophoma, Monilia, Candida, and Saccharomyces.

26. The method according to claim 22, wherein said rickettsiae microbe is selected from the group consisting of Coxiella burnetti, Bartonella quintana, Rochlimea Quintana, Rickettsia Quintana, Rickettsia prowasecki, and Rickettsia rickettsii.

27. The method according to claim 1, wherein said high-risk environment is selected from the group consisting of a retail food industry facility, a school, a medical environment, a water facility, a residence, a food transport vehicle, a processing facility, and a research facility.

28. The method according to claim 27, wherein said retail food industry facility is selected from the group consisting of a butcher shop, a grocery store, a restaurant, a cafeteria, an entertainment facility, and a convenience store.

29. The method according to claim 28, wherein said entertainment facility is selected from the group consisting of a theater, a park, a zoo, a rink, an arena, a civic center, a museum, and a stadium.

30. The method according to claim 27, wherein said medical environment is selected from the group consisting of a hospital, a physician's office, a dental office, a clinic, a nursing home, an outpatient facility, a physical therapy facility, a spa, an operating room, and a medical diagnostic laboratory.

31. The method according to claim 27, wherein said water facility is selected from the group consisting of a wastewater treatment plant, a potable water facility, a desalinization facility, a recycled water facility, an aquaculture facility, an air conditioning unit, a humidifier, a water storage tank, a water fountain, a fire hydrant, a tub, a hot tub, a sauna, a steam bath, and a water tap.

32. The method according to claim 27, wherein said food transport vehicle is selected from the group consisting of a truck, a rail car, and a ship.

33. The method according to claim 27, wherein said processing facility is selected from a food processing facility, a chemical processing facility, and a biological processing facility.

34. The method according to claim 33, wherein said food processing facility is selected from the group consisting of an abbatoir, a packaging facility, a purification plant, and a fermentation vessel.

35. The method according to claim 1, wherein said sample is a tissue sample.

36. The method according to claim 35, wherein said tissue sample is a biopsy sample.

37. The method according to claim 35, wherein said tissue sample is derived from a swab of an animal.

38. The method according to claim 37, wherein said animal is selected from the group consisting of a human, a cow, a pig, a horse, a goat, a sheep, a dog, a cat, a bird, a monkey, a fish, a clam, an oyster, a mussel, a lobster, a shrimp, and a crab.

39. The method according to claim 38, wherein said tissue sample is selected from the group consisting of an eye, a tongue, a cheek, a hoof, a beak, a snout, a foot, a hand, a mouth, a teat, the gastrointestinary tract, a feather, an ear, a nose, a mucous membrane, a scale, a shell, the fur, and the skin.

40. The method according to claim 1, wherein said sample comprises a fomite.

41. The method according to claim 1, wherein said sample is derived from a fomite present in said high-risk environment.

42. The method according to claim 1, wherein said sample is a food sample.

43. The method according to claim 42, wherein said food sample is a prepared food sample.

44. The method according to claim 42, wherein said food sample is raw.

45. The method according to claim 42, wherein said food sample is cooked.

46. The method according to claim 42, wherein said food sample is perishable.

47. The method according to claim 42, wherein said food sample is selected from the group consisting of beef, pork, poultry, seafood, dairy, fruit, vegetable, seed, nut, and fungus.

48. The method according to claim 47, wherein said dairy food sample is selected from the group consisting of milk, eggs, and cheese.

49. The method according to claim 1, wherein said sample is a liquid sample.

50. The method according to claim 49, wherein said liquid sample is selected from the group consisting of a water sample, a blood sample, a urine sample, a lachrymal sample, a sweat sample, a saliva sample, a lymph sample, and a cerebrospinal fluid sample.

51. An article of manufacture, comprising:
at least one cpn60 antibody, wherein said cpn60 antibody is attached to a solid support; and
an indicator molecule.

52. The article of manufacture of claim 51, further comprising instructions for using said cpn60 antibody to detect a cpn60-containing microbe.

53. The article of manufacture of claim 51, wherein said solid support is a dipstick.
